SEQUENCE LISTING (48 sequences)

SEQ ID NO: 1 - DNA, 360 bp, synthetic construct
  source 1..360: mol_type = other DNA
caggtgcagctggtggagtctgggggagacgtggtccagcctgggaggtccctgagactctcctgtgcagcctctggattcaccttcagttactatggcatgcactgggtccgccagcctccaggcaaggggctggagtgggtgacaattatatcatttgatggaagtcaaaaatactatgcagactccgtgaagggccgcttcaccatctccagagacaattccaagaacacggtttttttgcaaatgaacagcctgagaacagaggacacgggtttctattactgtgcggcccaaagtagtacctggcccgaatacttccagtactggggccagggcaccctggtcaccgtctcctca

SEQ ID NO: 2 - protein, 120 aa, synthetic construct
QVQLVESGGDVVQPGRSLRLSCAASGFTFSYYGMHWVRQPPGKGLEWVTIISFDGSQKYYADSVKGRFTISRDNSKNTVFLQMNSLRTEDTGFYYCAAQSSTWPEYFQYWGQGTLVTVSS

SEQ ID NO: 3 - DNA, 24 bp, synthetic construct
  source 1..24: mol_type = other DNA
ggattcaccttcagttactatggc

SEQ ID NO: 4 - protein, 8 aa, synthetic construct
GFTFSYYG

SEQ ID NO: 5 - DNA, 24 bp, synthetic construct
  source 1..24: mol_type = other DNA
atatcatttgatggaagtcaaaaa

SEQ ID NO: 6 - protein, 8 aa, synthetic construct
ISFDGSQK

SEQ ID NO: 7 - DNA, 39 bp, synthetic construct
  source 1..39: mol_type = other DNA
gcggcccaaagtagtacctggcccgaatacttccagtac

SEQ ID NO: 8 - protein, 13 aa, synthetic construct
AAQSSTWPEYFQY

SEQ ID NO: 9 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 30 bp, synthetic construct
  source 1..30: mol_type = other DNA
caacagagttacagtacccctccgatcacc

SEQ ID NO: 16 - protein, 10 aa, synthetic construct
QQSYSTPPIT

SEQ ID NO: 17 - DNA, 1344 bp, synthetic construct
  source 1..1344: mol_type = other DNA
caggtgcagctggtggagtctgggggagacgtggtccagcctgggaggtccctgagactctcctgtgcagcctctggattcaccttcagttactatggcatgcactgggtccgccagcctccaggcaaggggctggagtgggtgacaattatatcatttgatggaagtcaaaaatactatgcagactccgtgaagggccgcttcaccatctccagagacaattccaagaacacggtttttttgcaaatgaacagcctgagaacagaggacacgggtttctattactgtgcggcccaaagtagtacctggcccgaatacttccagtactggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 447 aa, synthetic construct
QVQLVESGGDVVQPGRSLRLSCAASGFTFSYYGMHWVRQPPGKGLEWVTIISFDGSQKYYADSVKGRFTISRDNSKNTVFLQMNSLRTEDTGFYYCAAQSSTWPEYFQYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 351 bp, synthetic construct
  source 1..351: mol_type = other DNA
caggtgcagctggtggagtctgggggaggcgtggtccagcctgggaggtccctgagactctcctgtgtagcgtctggattcaccttcagtacctctgtcatgcactgggtccgccaggctccaggcaagggactggaatgggtggcaaatatatggtatgatggaatcaataaattctatgtagactctgtgaaggggcgattcaccatctccagagacaattccaagaacaccctgtatctgcaaatgaacagcctgagagccgaggacacggctgtatattactgtgcgagagagttttggagtgcctttgatctctggggccaagggacaatggtcaccgtctcttca

SEQ ID NO: 22 - protein, 117 aa, synthetic construct
QVQLVESGGGVVQPGRSLRLSCVASGFTFSTSVMHWVRQAPGKGLEWVANIWYDGINKFYVDSVKGRFTISRDNSKNTLYLQMNSLRAEDTAVYYCAREFWSAFDLWGQGTMVTVSS

SEQ ID NO: 23 - DNA, 24 bp, synthetic construct
  source 1..24: mol_type = other DNA
ggattcaccttcagtacctctgtc

SEQ ID NO: 24 - protein, 8 aa, synthetic construct
GFTFSTSV

SEQ ID NO: 25 - DNA, 24 bp, synthetic construct
  source 1..24: mol_type = other DNA
atatggtatgatggaatcaataaa

SEQ ID NO: 26 - protein, 8 aa, synthetic construct
IWYDGINK

SEQ ID NO: 27 - DNA, 30 bp, synthetic construct
  source 1..30: mol_type = other DNA
gcgagagagttttggagtgcctttgatctc

SEQ ID NO: 28 - protein, 10 aa, synthetic construct
AREFWSAFDL

SEQ ID NO: 29 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 30 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 31 - DNA, 21 bp, synthetic construct
  source 1..21: mol_type = other DNA
cagtcagtctctagctcttat

SEQ ID NO: 32 - protein, 7 aa, synthetic construct
QSVSSSY

SEQ ID NO: 33-34 - skipped (2 entries)

SEQ ID NO: 35 - DNA, 27 bp, synthetic construct
  source 1..27: mol_type = other DNA
caacagtacggaagcagcccgtggacg

SEQ ID NO: 36 - protein, 9 aa, synthetic construct
QQYGSSPWT

SEQ ID NO: 37 - DNA, 1335 bp, synthetic construct
  source 1..1335: mol_type = other DNA
caggtgcagctggtggagtctgggggaggcgtggtccagcctgggaggtccctgagactctcctgtgtagcgtctggattcaccttcagtacctctgtcatgcactgggtccgccaggctccaggcaagggactggaatgggtggcaaatatatggtatgatggaatcaataaattctatgtagactctgtgaaggggcgattcaccatctccagagacaattccaagaacaccctgtatctgcaaatgaacagcctgagagccgaggacacggctgtatattactgtgcgagagagttttggagtgcctttgatctctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 444 aa, synthetic construct
QVQLVESGGGVVQPGRSLRLSCVASGFTFSTSVMHWVRQAPGKGLEWVANIWYDGINKFYVDSVKGRFTISRDNSKNTLYLQMNSLRAEDTAVYYCAREFWSAFDLWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 648 bp, synthetic construct
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 372 aa, synthetic construct
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 42 - protein, 348 aa, synthetic construct
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSEQKLISEEDLGGEQKLISEEDLHHHHHH

SEQ ID NO: 43 - protein, 348 aa, synthetic construct
QRHSPQEAPHVQYERLGSDVTLPCGTANWDAAVTWRVNGTDLAPDLLNGSQLVLRGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQIPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSEQKLISEEDLGGEQKLISEEDLHHHHHH

SEQ ID NO: 44 - protein, 348 aa, synthetic construct
QKHSPQEAPHVQYERLGADVTLPCGTASWDAAVTWRVNGTDLAPDLLNGSQLILRSLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNTTAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAPETTTSTTSSLAPPPTTKICDPGELGSEQKLISEEDLGGEQKLISEEDLHHHHHH

SEQ ID NO: 45 - protein, 553 aa, synthetic construct
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 46 - protein, 547 aa, synthetic construct
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 348 aa, synthetic construct
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNTTAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAPETTTSTTSSLAPPPTTKICDPGELGSEQKLISEEDLGGEQKLISEEDLHHHHHH

SEQ ID NO: 48 - protein, 348 aa, synthetic construct
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAPETTTSTTSSLAPPPTTKICDPGELGSEQKLISEEDLGGEQKLISEEDLHHHHHH

(19) United States
(12) Patent Application Publication
(10) Pub. No.: US 2026/0201045 A1
(43) Pub. Date: Jul. 16, 2026

(54) ANTIBODIES TO CILIARY NEUROTROPHIC FACTOR RECEPTOR (CNTFR) AND METHODS OF USE THEREOF

(71) Applicant: Regeneron Pharmaceuticals, Inc., Tarrytown, NY (US)

(72) Inventors: Judith Yu ALTAREJOS, Armonk, NY (US); Ashok T. BADITHE, Warren, NJ (US); Tammy T. HUANG, Cross River, NY (US); Jee Hae KIM, Ardsley, NY (US); Ashique RAFIQUE, Yonkers, NY (US); Yonaton Sholom RAY, Bergenfield, NJ (US); Panayiotis Elefterios STEVIS, Glenmoore, PA (US)

(21) Appl. No.: 18/993,314

(22) PCT Filed: Jul. 11, 2023
(86) PCT No.: PCT/US2023/069995

§ 371 Date: Jan. 10, 2025

Related U.S. Application Data
(60) Provisional application No. 63/368,201, filed on Jul. 12, 2022.

(51) Int. Cl. C07K 16/28

(52) U.S. Cl. CPC C07K 16/2866; C07K 2317/21; C07K 2317/33; C07K 2317/565; C07K 2317/76; C07K 2317/92

(57) ABSTRACT

The present disclosure provides antibodies and antigen-binding fragments thereof that bind to ciliary neurotrophic factor receptor (CNTFR). Methods for using anti-CNTFR antibodies and antigen-binding fragments are also provided.

### FIELD OF THE INVENTION

The present disclosure relates to antibodies and antigen-binding fragments of human antibodies that specifically bind to ciliary neurotrophic factor receptor (CNTFR), and methods of use thereof.

### SEQUENCE

An official copy of the sequence listing is submitted concurrently with the specification electronically via USPTO Patent Center as an XML formatted sequence. The contents of the electronic sequence listing (11225WO01_Sequence_Listing_ST26.xml.; Size: 53,248 bytes; and Date of Creation: Jul. 11, 2023) is herein incorporated by reference in its entirety.

### BACKGROUND

Ciliary neurotrophic factor (CNTF) is a cytokine of the interleukin-6 (IL-6) family that enhances survival and differentiation of neurons in the central nervous system and peripheral nervous system (Purser et al., PLoS ONE 2013, 8: e61616). CNTF also induces weight loss and improves glucose tolerance in humans and rodents (Watt et al., Nat Med 2006, 12:541-548). CNTF binds to the CNTF receptor alpha subunit (CNTFRα), which is a member of the class I hematopoietin receptor family that contains an N-terminal immunoglobulin (Ig)-like module and a cytokine binding domain (CBD) formed from two fibronectin type III (FNIII) modules linked by a proline-rich sequence (Man et al., J Biol Chem 2003, 278: P23285-23294). CNTF then signals via gp130 and leukemia inhibitory factor receptor (LIFR) (Davis et al., Science 1991, 253:59-61; Davis et al., Science 1993, 260:1805-1808). However, the overall architecture of the CNTF signaling complex is not well characterized.

CNTF and CNTFRα are expressed in various tissues throughout the central nervous system and peripheral nervous system, such as in retinal cells, glial cells, and dorsal root ganglion (DRG) neurons (see, e.g., Sleeman et al., Pharm Acta Helv 2000, 74:265-272; Beltran et al., Molecular Vision 2005, 11:232-244). CNTF has been shown to induce neuronal cell differentiation and neural outgrowth and exert neuroprotective effects, including in retinal cells, DRG neurons, and oligodendrocytes (see, e.g., Leibinger et al., J Neurosci 2009, 29:14334-14341; Wen et al., Neural Regeneration Research 2017, 12:1716-1723; and Perugini et al., Nature Sci Reports 2022, 12:8331). CNTF and CNTFRα accordingly are attractive targets for the treatment of neurological, neurodegenerative, and retinal diseases.

CNTFRα is also widely expressed in metabolically important organs and cell types such as skeletal muscle fibers, white and brown adipocytes, hepatocytes, pancreatic islet cells, and intestinal cells (see Perugini et al., Nature Sci Reports 2022, 12:8331). Administration of CNTF or modified variants of CNTF in animal models of obesity has been shown to reduce body weight, reduce glycemia, and attenuate hyperinsulinemia (Battista et al., PLoS ONE 2022, 17 (3): e0265749), and patients administered the recombinant human variant CNTF Axokine achieved weight loss in clinical trials (Ettinger et al., JAMA 2003, 289:1826-1832).

### SUMMARY OF THE INVENTION

In one aspect, isolated antibodies or antigen-binding fragment thereof that specifically bind to ciliary neurotrophic factor receptor (CNTFR) are provided. In some embodiments, the antibody or antigen-binding fragment thereof specifically binds to human CNTFR.

In some embodiments, the antibody or antigen-binding fragment thereof binds to dimeric human CNTFR with a K_{D} of less than 10 nM as measured by surface plasmon resonance at 25° C. In some embodiments, the antibody or antigen-binding fragment thereof (i) binds an epitope in the region of Q23-V105 of human CNTFR; (ii) binds an epitope in the region of V105-D205 of human CNTFR; (iii) cross-reacts with cynomolgus monkey CNTFR; and/or (iv) is a fully human monoclonal antibody.

In some embodiments, the antibody or antigen-binding fragment thereof comprises:
(a) a heavy chain complementarity determining region 1 (HCDR1) comprising an amino acid sequence selected from the group consisting of SEQ ID NOs: 4 and 24;
(b) a heavy chain complementarity determining region 2 (HCDR2) comprising an amino acid sequence selected from the group consisting of SEQ ID NOs: 6 and 26;
(c) a heavy chain complementarity determining region 3 (HCDR3) comprising an amino acid sequence selected from the group consisting of SEQ ID NOs: 8 and 28;
(d) a light chain complementarity determining region 1 (LCDR1) comprising an amino acid sequence selected from the group consisting of SEQ ID NOs: 12 and 32;
(e) a light chain complementarity determining region 2 (LCDR2) comprising an amino acid sequence selected from the group consisting of SEQ ID NOs: 14 and 34; and
(f) a light chain complementarity determining region 3 (LCDR3) comprising an amino acid sequence selected from the group consisting of SEQ ID NOs: 16 and 36.

In some embodiments, the antibody or antigen-binding fragment thereof comprises:
(a) an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence of SEQ ID NO:14, and an LCDR3 comprising the amino acid sequence of SEQ ID NO:16; or
(b) an HCDR1 comprising the amino acid sequence of SEQ ID NO:24, an HCDR2 comprising the amino acid sequence of SEQ ID NO:26, an HCDR3 comprising the amino acid sequence of SEQ ID NO:28, an LCDR1 comprising the amino acid sequence of SEQ ID NO:32, an LCDR2 comprising the amino acid sequence of SEQ ID NO:34, and an LCDR3 comprising the amino acid sequence of SEQ ID NO:36.

In some embodiments, the antibody or antigen-binding fragment thereof comprises a heavy chain variable region (HCVR) comprising an amino acid sequence that has at least 85% sequence identity to an amino acid sequence selected from the group consisting of SEQ ID NOs: 2 and 22. In some embodiments, the antibody or antigen-binding fragment thereof comprises a light chain variable region (LCVR) comprising an amino acid sequence that has at least 85% sequence identity to an amino acid sequence selected from the group consisting of SEQ ID NOs: 10 and 30.

In some embodiments, the antibody or antigen-binding fragment thereof comprises:
(a) an HCVR comprising the amino acid sequence having at least 85%, at least 90%, at least 95%, or 100% sequence identity to the amino acid sequence of SEQ ID NO:2 and an LCVR comprising the amino acid sequence having at least 85%, at least 90%, at least 95%, at least 97%, at least 98%, at least 99%, or 100% sequence identity to the amino acid sequence of SEQ ID NO:10; or
(b) an HCVR comprising the amino acid sequence having at least 85%, at least 90%, at least 95%, at least 97%, at least 98%, at least 99%, or 100% sequence identity to the amino acid sequence of SEQ ID NO:22 and an LCVR comprising the amino acid sequence having at least 85%, at least 90%, at least 95%, at least 97%, at least 98%, at least 99%, or 100% sequence identity to the amino acid sequence of SEQ ID NO:30.

In some embodiments, the antibody or antigen-binding fragment thereof comprises:
(a) an HCVR comprising the amino acid sequence having at least 85% sequence identity to the amino acid sequence of SEQ ID NO:2 and an LCVR comprising the amino acid sequence having at least 85% sequence identity to the amino acid sequence of SEQ ID NO:10; or
(b) an HCVR comprising the amino acid sequence having at least 85% sequence identity to the amino acid sequence of SEQ ID NO:22 and an LCVR comprising the amino acid sequence having at least 85% sequence identity to the amino acid sequence of SEQ ID NO:30.

In some embodiments, the antibody or antigen-binding fragment thereof comprises:
(a) an HCVR comprising the amino acid sequence having at least 90% sequence identity to the amino acid sequence of SEQ ID NO:2 and an LCVR comprising the amino acid sequence having at least 90% sequence identity to the amino acid sequence of SEQ ID NO:10; or
(b) an HCVR comprising the amino acid sequence having at least 90% sequence identity to the amino acid sequence of SEQ ID NO:22 and an LCVR comprising the amino acid sequence having at least 90% sequence identity to the amino acid sequence of SEQ ID NO:30.

In some embodiments, the antibody or antigen-binding fragment thereof comprises:
(a) an HCVR comprising the amino acid sequence having at least 95% sequence identity to the amino acid sequence of SEQ ID NO:2 and an LCVR comprising the amino acid sequence having at least 95% sequence identity to the amino acid sequence of SEQ ID NO:10; or
(b) an HCVR comprising the amino acid sequence having at least 95% sequence identity to the amino acid sequence of SEQ ID NO:22 and an LCVR comprising the amino acid sequence having at least 95% sequence identity to the amino acid sequence of SEQ ID NO:30.

In some embodiments, the antibody or antigen-binding fragment thereof comprises:
(a) an HCVR comprising the amino acid sequence of SEQ ID NO:2 and an LCVR comprising the amino acid sequence of SEQ ID NO:10; or
(b) an HCVR comprising the amino acid sequence of SEQ ID NO:22 and an LCVR comprising the amino acid sequence of SEQ ID NO:30.

In some embodiments, the antibody or antigen-binding fragment thereof comprises:
(a) a heavy chain comprising an amino acid sequence having at least 85% sequence identity to the amino acid sequence of SEQ ID NO:18 and a light chain comprising an amino acid sequence having at least 85% sequence identity to the amino acid sequence of SEQ ID NO: 20; or
(b) a heavy chain comprising an amino acid sequence having at least 85% sequence identity to the amino acid sequence of SEQ ID NO:38 and a light chain comprising an amino acid sequence having at least 85% sequence identity to the amino acid sequence of SEQ ID NO: 40.

In some embodiments, the antibody or antigen-binding fragment thereof comprises:
(a) a heavy chain comprising an amino acid sequence having at least 90% sequence identity to the amino acid sequence of SEQ ID NO:18 and a light chain comprising an amino acid sequence having at least 90% sequence identity to the amino acid sequence of SEQ ID NO: 20; or
(b) a heavy chain comprising an amino acid sequence having at least 90% sequence identity to the amino acid sequence of SEQ ID NO:38 and a light chain comprising an amino acid sequence having at least 90% sequence identity to the amino acid sequence of SEQ ID NO: 40.

In some embodiments, the antibody or antigen-binding fragment thereof comprises:
(a) a heavy chain comprising an amino acid sequence having at least 95% sequence identity to the amino acid sequence of SEQ ID NO:18 and a light chain comprising an amino acid sequence having at least 95% sequence identity to the amino acid sequence of SEQ ID NO: 20; or
(b) a heavy chain comprising an amino acid sequence having at least 95% sequence identity to the amino acid sequence of SEQ ID NO:38 and a light chain comprising an amino acid sequence having at least 95% sequence identity to the amino acid sequence of SEQ ID NO: 40.

In some embodiments, the antibody or antigen-binding fragment thereof comprises:
(a) a heavy chain comprising the amino acid sequence of SEQ ID NO:18 and a light chain comprising the amino acid sequence of SEQ ID NO:20; or
(b) a heavy chain comprising the amino acid sequence of SEQ ID NO:38 and a light chain comprising the amino acid sequence of SEQ ID NO:40.

In another aspect, pharmaceutical compositions are provided, comprising an anti-CNTFR antibody or antigen-binding fragment as described herein, along with a pharmaceutically acceptable carrier.

In another aspect, nucleic acid molecules comprising a nucleotide sequence encoding an anti-CNTFR antibody or antigen-binding fragment thereof as described herein are provided. In some embodiments, the nucleic acid molecule comprises one or more nucleotide sequences set forth in Table 14.

In some embodiments, the isolated nucleic acid molecule comprises a nucleic acid sequence encoding a heavy chain variable region (HCVR) of an antibody that binds CNTFR, wherein the HCVR comprises a heavy chain complementarity determining region 1 (HCDR1) comprising SEQ ID NO: 4, a heavy chain complementarity determining region 2 (HCDR2) comprising SEQ ID NO: 6, and a heavy chain complementarity determining region 3 (HCDR3) comprising SEQ ID NO: 8.

In some embodiments, the isolated nucleic acid molecule comprises a nucleic acid sequence encoding a heavy chain variable region (HCVR) of an antibody that binds CNTFR, wherein the HCVR comprises a heavy chain complementarity determining region 1 (HCDR1) comprising SEQ ID NO: 24, a heavy chain complementarity determining region 2 (HCDR2) comprising SEQ ID NO: 26, and a heavy chain complementarity determining region 3 (HCDR3) comprising SEQ ID NO: 28.

In some embodiments, the isolated nucleic acid molecule comprises a nucleic acid sequence encoding a heavy chain variable region (HCVR) of an antibody that binds CNTFR and the HCVR comprises the amino acid sequence of SEQ ID NO: 2.

In some embodiments, the isolated nucleic acid molecule comprises a nucleic acid sequence encoding a heavy chain variable region (HCVR) of an antibody that binds CNTFR and the HCVR comprises the amino acid sequence of SEQ ID NO: 22.

In some embodiments, the HCVR comprises an HCDR1 encoded by the nucleotide sequence of SEQ ID NO:3, an HCDR2 encoded by the nucleotide sequence of SEQ ID NO:5, and an HCDR3 encoded by the nucleotide sequence of SEQ ID NO: 7.

In some embodiments, the HCVR comprises an HCDR1 encoded by the nucleotide sequence of SEQ ID NO: 23, an HCDR2 encoded by the nucleotide sequence of SEQ ID NO: 25, and an HCDR3 encoded by the nucleotide sequence of SEQ ID NO: 27.

In some embodiments, the nucleic acid molecule comprises the nucleotide sequence of SEQ ID NO: 1, or a substantially identical sequence having at least 95% homology thereto.

In some embodiments, the nucleic acid molecule comprises the nucleotide sequence of SEQ ID NO: 21, or a substantially identical sequence having at least 95% homology thereto.

In some embodiments, the nucleic acid molecule comprises the nucleotide sequence of SEQ ID NO: 17 or a substantially identical sequence having at least 95% homology thereto.

In some embodiments, the nucleic acid molecule comprises the nucleotide sequence of SEQ ID NO: 17.

In some embodiments, the nucleic acid molecule comprises the nucleotide sequence of SEQ ID NO: 37 or a substantially identical sequence having at least 95% homology thereto.

In some embodiments, the nucleic acid molecule comprises the nucleotide sequence of SEQ ID NO: 37.

In some embodiments, the isolated nucleic acid molecule comprises a nucleic acid sequence encoding a light chain variable region (LCVR) of an antibody that binds CNTFR, wherein the LCVR comprises a light chain complementarity determining region 1 (LCDR1) comprising SEQ ID NO: 12, a light chain complementarity determining region 2 (LCDR2) comprising SEQ ID NO: 14, and a light chain complementarity determining region 3 (LCDR3) comprising SEQ ID NO: 16.

In some embodiments, the isolated nucleic acid molecule comprises a nucleic acid sequence encoding a light chain variable region (LCVR) of an antibody that binds CNTFR, wherein the LCVR comprises a light chain complementarity determining region 1 (LCDR1) comprising SEQ ID NO: 32, a light chain complementarity determining region 2 (LCDR2) comprising SEQ ID NO: 34, and a light chain complementarity determining region 3 (LCDR3) comprising SEQ ID NO: 36.

In some embodiments, the isolated nucleic acid molecule comprises a nucleic acid sequence encoding a light chain variable region (LCVR) of an antibody that binds CNTFR and the LCVR comprises SEQ ID NO: 10.

In some embodiments, the isolated nucleic acid molecule comprises a nucleic acid sequence encoding a light chain variable region (LCVR) of an antibody that binds CNTFR and the LCVR comprises SEQ ID NO: 30.

In some embodiments, the LCVR comprises an LCDR1 encoded by the nucleotide sequence of SEQ ID NO: 11, an LCDR2 encoded by the nucleotide sequence of SEQ ID NO: 13, and an LCDR3 encoded by the nucleotide sequence of SEQ ID NO: 15.

The nucleic acid molecule of claim 6, wherein the LCVR comprises an LCDR1 encoded by the nucleotide sequence of SEQ ID NO: 31, an LCDR2 encoded by the nucleotide sequence of SEQ ID NO:33, and an LCDR3 encoded by the nucleotide sequence of SEQ ID NO: 35.

In some embodiments, the nucleic acid molecule comprises the nucleotide sequence of SEQ ID NO: 9, or a substantially identical sequence having at least 95% homology thereto.

In some embodiments, the nucleic acid molecule comprises the nucleotide sequence of SEQ ID NO: 29, or a substantially identical sequence having at least 95% homology thereto.

In some embodiments, the nucleic acid molecule comprises the nucleotide sequence of SEQ ID NO: 19 or a substantially identical sequence having at least 95% homology thereto.

In some embodiments, the nucleic acid molecule comprises the nucleotide sequence of SEQ ID NO: 19.

In some embodiments, the nucleic acid molecule comprises the nucleotide sequence of SEQ ID NO: 39 or a substantially identical sequence having at least 95% homology thereto.

In some embodiments, the nucleic acid molecule comprises the nucleotide sequence of SEQ ID NO: 39.

In some aspects, provided herein is an expression vector comprising:
(a) a nucleic acid molecule comprising a nucleic acid sequence encoding a heavy chain complementarity determining region 1 (HCDR1), a heavy chain complementarity determining region 2 (HCDR2), and a heavy chain complementarity determining region 3 (HCDR3) within a heavy chain variable region (HCVR) amino acid sequence of SEQ ID NO: 2; and/or
(b) a nucleic acid molecule comprising a nucleic acid sequence encoding a light chain complementarity determining region 1 (LCDR1), a light chain complementarity determining region 2 (LCDR2), and a light chain complementarity determining region 3 (LCDR3) within a light chain variable region (LCVR) amino acid sequence of SEQ ID NO: 10.

In some aspects, provided herein is an expression vector comprising:
(a) a nucleic acid molecule comprising a nucleic acid sequence encoding a heavy chain variable region (HCVR) of an antibody that binds CNTFR, wherein the HCVR comprises a heavy chain complementarity determining region 1 (HCDR1) comprising SEQ ID NO: 4, a heavy chain complementarity determining region 2 (HCDR2) comprising SEQ ID NO: 6, and a heavy chain complementarity determining region 3 (HCDR3) comprising SEQ ID NO: 8; and/or
(b) a nucleic acid molecule comprising a nucleic acid sequence encoding a light chain variable region (LCVR) of an antibody that binds CNTFR, wherein the LCVR comprises a light chain complementarity determining region 1 (LCDR1) comprising SEQ ID NO: 12, a light chain complementarity determining region 2 (LCDR2) comprising SEQ ID NO: 14, and a light chain complementarity determining region 3 (LCDR3) comprising SEQ ID NO: 16.

In some aspects, provided herein is an expression vector comprising:
(a) a nucleic acid molecule comprising a nucleic acid sequence encoding a heavy chain variable region (HCVR) of an antibody that binds CNTFR, wherein the HCVR comprises at least 85%, at least 90%, at least 95%, or 100% sequence identity to the amino acid sequence of SEQ ID NO: 2. In some embodiments, the nucleic acid molecule comprises a nucleic acid sequence encoding an HCVR of SEQ ID NO: 2; and/or
(b) a nucleic acid molecule comprising a nucleic acid sequence encoding a light chain variable region (LCVR) of an antibody that binds CNTFR, wherein the LCVR comprises at least 85%, at least 90%, at least 95%, or 100% sequence identity to the amino acid sequence of SEQ ID NO: 10. In some embodiments, the nucleic acid molecule comprises a nucleic acid sequence encoding an LCVR of SEQ ID NO: 10.

In some aspects, provided herein is an expression vector comprising:
(a) a nucleic acid molecule comprising a nucleic acid sequence encoding a heavy chain complementarity determining region 1 (HCDR1), a heavy chain complementarity determining region 2 (HCDR2), and a heavy chain complementarity determining region 3 (HCDR3) within a heavy chain variable region (HCVR) amino acid sequence of SEQ ID NO: 22; and/or
(b) a nucleic acid molecule comprising a nucleic acid sequence encoding a light chain complementarity determining region 1 (LCDR1), a light chain complementarity determining region 2 (LCDR2), and a light chain complementarity determining region 3 (LCDR3) within a light chain variable region (LCVR) amino acid sequence of SEQ ID NO: 30.

In some aspects, provided herein is an expression vector comprising:
(a) a nucleic acid molecule comprising a nucleic acid sequence encoding a heavy chain variable region (HCVR) of an antibody that binds CNTFR, wherein the HCVR comprises a heavy chain complementarity determining region 1 (HCDR1) comprising SEQ ID NO: 24, a heavy chain complementarity determining region 2 (HCDR2) comprising SEQ ID NO: 26, and a heavy chain complementarity determining region 3 (HCDR3) comprising SEQ ID NO: 28; and/or
(b) a nucleic acid molecule comprising a nucleic acid sequence encoding a light chain variable region (LCVR) of an antibody that binds CNTFR, wherein the LCVR comprises a light chain complementarity determining region 1 (LCDR1) comprising SEQ ID NO: 32, a light chain complementarity determining region 2 (LCDR2) comprising SEQ ID NO: 34, and a light chain complementarity determining region 3 (LCDR3) comprising SEQ ID NO: 36.

In some aspects, provided herein is an expression vector comprising:
(a) a nucleic acid molecule comprising a nucleic acid sequence encoding a heavy chain variable region (HCVR) of an antibody that binds CNTFR, wherein the HCVR comprises at least 85%, at least 90%, at least 95%, or 100% sequence identity to the amino acid sequence of SEQ ID NO: 22. In some embodiments, the nucleic acid molecule comprises a nucleic acid sequence encoding an HCVR of SEQ ID NO: 22, and/or
(b) a nucleic acid molecule comprising a nucleic acid sequence encoding a light chain variable region (LCVR) of an antibody that binds CNTFR, wherein the LCVR comprises at least 85%, at least 90%, at least 95%, or 100% sequence identity to the amino acid sequence of SEQ ID NO: 30. In some embodiments, the nucleic acid molecule comprises a nucleic acid sequence encoding an LCVR of SEQ ID NO: 30.

In some embodiments, an isolated host cell comprises an expression vector encoding a heavy chain variable region (HCVR) as set forth in SEQ ID NO: 2. In some embodiments, an isolated host cell comprises an expression vector encoding a light chain variable region (LCVR) as set forth in SEQ ID NO: 10. In some embodiments, an isolated host cell comprises an expression vector encoding an HCVR as set forth in SEQ ID NO: 2 and an LCVR as set forth in SEQ ID NO: 10.

In some embodiments, an isolated host cell comprises an expression vector encoding a heavy chain variable region (HCVR) as set forth in SEQ ID NO: 22. In some embodiments, an isolated host cell comprises an expression vector encoding a light chain variable region (LCVR) as set forth in SEQ ID NO: 30. In some embodiments, an isolated host cell comprises an expression vector encoding an HCVR as set forth in SEQ ID NO: 22 and an LCVR as set forth in SEQ ID NO: 30.

In some aspects, the host cell is a mammalian cell or a prokaryotic cell.

In some aspects, the host cell is a Chinese Hamster Ovary (CHO) cell or an Escherichia coli (E. coli) cell.

Provided herein are methods of producing an anti-CNTFR antibody or antigen-binding fragment thereof, the method comprising growing a host cell provided herein under conditions permitting production of the antibody or antigen-binding fragment thereof, wherein said host cell comprises both a nucleic acid molecule comprising a nucleic acid sequence encoding said HCVR or heavy chain as provided above and a nucleic acid molecule comprising a nucleic acid sequence encoding said LCVR or light chain as provided above.

Also provided herein are compositions comprising a first nucleic acid molecule and a second nucleic acid molecule;
wherein the first nucleic acid molecule comprises a nucleic acid sequence encoding a heavy chain variable region (HCVR) of an anti-CNTFR antibody that comprises a heavy chain complementarity determining region 1 (HCDR1) comprising SEQ ID NO: 4, a heavy chain complementarity determining region 2 (HCDR2) comprising SEQ ID NO: 6, and a heavy chain complementarity determining region 3 (HCDR3) comprising SEQ ID NO: 8; and
wherein the second nucleic acid molecule comprises a nucleic acid sequence encoding a light chain variable region (LCVR) of an anti-CNTFR antibody that comprises a light chain complementarity determining region 1 (LCDR1) comprising SEQ ID NO: 12, a light chain complementarity determining region 2 (LCDR2) comprising SEQ ID NO: 14, and a light chain complementarity determining region 3 (LCDR3) comprising SEQ ID NO: 16.

In some embodiments, the first nucleic acid molecule encodes an HCVR comprising SEQ ID NO: 2, and the second nucleic acid molecule encodes an LCVR comprising SEQ ID NO: 10.

In some embodiments, the first nucleic acid molecule encodes a heavy chain comprising SEQ ID NO: 18, and the second nucleic acid molecule encodes a light chain comprising SEQ ID NO: 20.

Also provided herein are compositions comprising a first nucleic acid molecule and a second nucleic acid molecule;
wherein the first nucleic acid molecule comprises a nucleic acid sequence encoding a heavy chain variable region (HCVR) of an anti-CNTFR antibody that comprises a heavy chain complementarity determining region 1 (HCDR1) comprising SEQ ID NO: 24, a heavy chain complementarity determining region 2 (HCDR2) comprising SEQ ID NO: 26, and a heavy chain complementarity determining region 3 (HCDR3) comprising SEQ ID NO: 28; and
wherein the second nucleic acid molecule comprises a nucleic acid sequence encoding a light chain variable region (LCVR) of an anti-CNTFR antibody that comprises a light chain complementarity determining region 1 (LCDR1) comprising SEQ ID NO: 32, a light chain complementarity determining region 2 (LCDR2) comprising SEQ ID NO: 34, and a light chain complementarity determining region 3 (LCDR3) comprising SEQ ID NO: 36.

In some embodiments, the first nucleic acid molecule encodes an HCVR comprising SEQ ID NO: 22, and the second nucleic acid molecule encodes an LCVR comprising SEQ ID NO: 30.

In some embodiments, the first nucleic acid molecule encodes a heavy chain comprising SEQ ID NO: 38, and the second nucleic acid molecule encodes a light chain comprising SEQ ID NO: 40.

Further provided herein is an isolated nucleic acid molecule encoding an antibody or antigen-binding fragment thereof, which specifically binds CNTFR, wherein the antibody or antigen-binding fragment comprises a heavy chain variable region (HCVR) comprising three heavy chain complementarity determining regions (HCDR1, HCDR2 and HCDR3) of SEQ ID NO: 2, and a light chain variable region (LCVR) comprising three light chain complementarity determining regions (LCDR1, LCDR2 and LCDR3) of SEQ ID NO: 10. In some embodiments, (a) the HCDR1 comprises SEQ ID NO: 4; (b) the HCDR2 comprises SEQ ID NO: 6; (c) the HCDR3 comprises SEQ ID NO: 8; (d) the LCDR1 comprises SEQ ID NO: 12; (e) the LCDR2 comprises SEQ ID NO: 14; and (f) the LCDR3 comprises SEQ ID NO: 16. In some embodiments, the HCVR comprises SEQ ID NO: 2. In some embodiments, the LCVR comprises SEQ ID NO: 10. In some embodiments, the HCVR comprises SEQ ID NO: 2 and the LCVR comprises SEQ ID NO: 10.

Also provided herein are expression vectors and host cells comprising a nucleic acid molecule as disclosed herein.

In another aspect, method of producing an anti-CNTFR antibody or antigen-binding fragment thereof are provided. In some embodiments, the method comprises introducing a nucleic acid molecule or expression vector encoding an anti-CNTFR antibody or antigen-binding fragment thereof as disclosed herein into a host cell, growing the host cell under conditions permitting production of the antibody or antigen-binding fragment thereof, and recovering the antibody or antigen-binding fragment that is produced.

In another aspect, methods of treating a disease, disorder, or condition associated with CNTFR expression or activity are provided. In some embodiments, the method comprises administering an anti-CNTFR antibody or antigen-binding fragment thereof or pharmaceutical composition as disclosed herein to a subject in need thereof. In some embodiments, the disease, disorder, or condition is a metabolic disease. In some embodiments, the disease, disorder, or condition is a neurological or neurodegenerative disease.

Provided herein is a method for inhibiting CNTF-mediated activation of cells expressing CNFTR, the method comprising contacting an anti-CNTFR antibody with the CNFTR-expressing cells. In some aspects, the anti-CNTFR antibody comprises:
(a) an HCVR comprising the amino acid sequence of SEQ ID NO:2 or a sequence that is 95% identical thereto and an LCVR comprising the amino acid sequence of SEQ ID NO:10 or a sequence that is 95% identical thereto; or
(b) an HCVR comprising the amino acid sequence of SEQ ID NO:22 or a sequence that is 95% identical thereto and an LCVR comprising the amino acid sequence of SEQ ID NO:30 or a sequence that is 95% identical thereto.

Other embodiments will be apparent from a review of the ensuing detailed description.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The term “CNTF receptor” or “CNTFR,” as used herein, refers to a receptor that binds ciliary neurotrophic factor (CNTF), also referred to herein as CNTF receptor alpha subunit (CNTFRα). As used herein, the term “CNTFR” may refer to a precursor form or a mature (i.e., processed) form or a fragment thereof. In some embodiments, the term “CNTFR” refers to a human CNTFR protein or fragment thereof. In some embodiments, the CNTFR protein comprises the amino acid sequence of human CNTFR set forth in Uniprot Accession No. P26992, or a fragment thereof (e.g., a proteolytically processed portion), or an isoform thereof. In some embodiments, a CNTFR protein has the sequence of SEQ ID NO:41. In some embodiments, a CNTFR protein is a fragment of SEQ ID NO:41, e.g., a portion comprising amino acids Q23-S342 of SEQ ID NO:41.

The term “antibody,” as used herein, refers to an antigen-binding molecule or molecular complex comprising a set of complementarity determining regions (CDRs) that specifically bind to or interact with a particular antigen (e.g., CNTFR). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, “antigen-binding domain,” and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for CNFTR or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant antibody,” as used herein, is intended to include all antibodies that are prepared, expressed, created or isolated by recombinant means. The term includes, but is not limited to, antibodies expressed using a recombinant expression vector transfected into a host cell (e.g., Chinese hamster ovary (CHO) cell) or cellular expression system, antibodies isolated from a recombinant, combinatorial human antibody library, and antibodies isolated from a non-human animal (e.g., a mouse, such as a mouse that is transgenic for human immunoglobulin genes (see e.g., Taylor et al. (1992) Nucl. Acids Res. 20:6287-6295). In some embodiments, the recombinant antibody is a recombinant human antibody. In some embodiments, recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less, e.g., 10⁻⁷ M, 10⁻⁸ M, 10⁻⁹ M, 10⁻¹⁰ M, 10⁻¹¹ M, or 10⁻¹² M (a smaller K_{D} denotes a tighter binding). Methods for determining whether an antibody specifically binds to an antigen are known in the art and include, for example, equilibrium dialysis, surface plasmon resonance (e.g., BIACORE™), bio-layer interferometry assay (e.g., Octet® HTX biosensor), solution-affinity ELISA, and the like. In some embodiments, specific binding is measured in a surface plasmon resonance assay, e.g., at 25° C. or 37° C. An antibody or antigen-binding fragment that specifically binds an antigen from one species may or may not have cross-reactivity to other antigens, such as an orthologous antigen from another species.

The term “K_{D},” as used herein, refers to the equilibrium dissociation constant of a particular antibody-antigen interaction.

The term “surface plasmon resonance,” as used herein, refers to an optical phenomenon that allows for the analysis of real-time biomolecular interactions by detection of alterations in protein concentrations within a biosensor matrix, for example using the BIACORE™ system (Cytiva, Marlborough, MA).

The term “bio-layer interferometry” or “BLI,” as used herein, refers to an optical technique for measuring real-time biomolecular interactions by analyzing interference patterns caused by the binding of one biomolecule dispensed in the sample plate to a second biomolecule immobilized on a biosensor plate, for example using the Octet® system (Sartorius AG, Göttingen, Germany).

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be either linear or discontinuous (e.g., conformational). A linear epitope is one produced by adjacent amino acid residues in a polypeptide chain. A conformational epitope is produced by spatially juxtaposed amino acids from different segments of the linear polypeptide chain. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes may also be defined as structural or functional. Functional epitopes are generally a subset of the structural epitopes and have those residues that directly contribute to the affinity of the interaction. An epitope typically includes at least 3, and more usually, at least 5 or at least 8-10 amino acids in a unique spatial conformation.

Methods for determining the epitope of an antigen-binding protein, e.g., an antibody or antigen-binding fragment, include alanine scanning mutational analysis, peptide blot analysis (Reineke, Methods Mol Biol 2004, 248:443-463), peptide cleavage analysis, crystallographic studies, and NMR analysis. In addition, methods such as epitope exclusion, epitope extraction, and chemical modification of antigens can be employed (Tomer, Prot Sci 2000, 9:487-496). Another method that can be used to identify the amino acids within a polypeptide with which an antigen-binding protein (e.g., an antibody or antigen-binding fragment) interacts is hydrogen/deuterium exchange detected by mass spectrometry (HDX). See, e.g., Ehring, Analytical Biochemistry 1999, 267:252-259; Engen and Smith, Anal Chem 2001, 73: 256A-265A.

The terms “substantial identity” and “substantially identical,” as used with reference to a nucleic acid or fragment thereof, indicates that, when optimally aligned with appropriate nucleotide insertions or deletions with another nucleic acid (or its complementary strand), there is nucleotide sequence identity in at least about 85%, e.g., at least 90%, 91%, 92%, 93%, 94%, 95%, 96%, 97%, 98% or 99%, of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the terms “substantial identity” and “substantially identical” mean that two peptide sequences, when optimally aligned, share at least about 85% sequence identity, e.g., at least 90%, 91%, 92%, 93%, 94%, 95%, 96%, 97%, 98% or 99% sequence identity. In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, 2000 supra). Another preferred algorithm when comparing a sequence of the disclosure to a database containing a large number of sequences from different organisms is the computer program BLAST, especially BLASTP or TBLASTN, using default parameters. (See, e.g., Altschul et al., 1990, J. Mol. Biol. 215:403-410 and 1997 Nucleic Acids Res. 25:3389-3402).

A “variant” of a polypeptide, such an immunoglobulin, VH, VL, heavy chain, light chain, or CDR comprising an amino acid sequence specifically set forth herein, refers to a polypeptide comprising an amino acid sequence that is at least about 70%-99.9% (e.g., at least 70, 75, 80, 85, 90, 91, 92, 93, 94, 95, 96, 97, 98, 99, 99.5, or 99.9%) identical to the reference polypeptide sequence (e.g., as set forth in the sequence listing below), when the comparison is performed by a BLAST algorithm wherein the parameters of the algorithm are selected to give the largest match between the respective sequences over the entire length of the respective reference sequences. In some embodiments, a variant of a polypeptide includes a polypeptide having the amino acid sequence of a reference polypeptide sequence (e.g., as set forth in the sequence listing below) but for one or more (e.g., 1 to 10, or less than 20, or less than 10) missense mutations (e.g., conservative substitutions), nonsense mutations, deletions, or insertions.

The term “therapeutically effective amount” refers to an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

### Anti-CNTFR Antibodies and Antigen-Binding Fragments Thereof

In one aspect, the present disclosure relates to antibodies and antigen-binding fragments thereof that bind to CNTFR. In some embodiments, the antibody or antigen-binding fragment binds an N-terminal Ig-like domain, or a portion thereof, in CNTFR. In some embodiments, the antibody or antigen-binding fragment binds an FNIII domain, or a portion thereof, in CNTFR. In some embodiments, the antibody or antigen-binding fragment thereof binds at least two domains or portions of at least two domains in CNTFR.

In some embodiments, the antibody or antigen-binding fragment thereof specifically binds to human CNTFR. In some embodiments, the antibody or antigen-binding fragment thereof specifically binds to human CNTFR and cross-reacts with CNTFR from one or more other non-human species, such as but not limited to mouse, rat, guinea pig, hamster, gerbil, pig, cat, dog, rabbit, goat, sheep, cow, horse, camel, cynomolgus, marmoset, rhesus or chimpanzee CNTFR. In some embodiments, the antibody or antigen-binding fragment thereof specifically binds to human CNTFR and cross-reacts with at least some (e.g., some but not all) non-human forms of CNTFR (e.g., mouse, rat, guinea pig, hamster, gerbil, pig, cat, dog, rabbit, goat, sheep, cow, horse, camel, cynomolgus, marmoset, rhesus or chimpanzee CNTFR). In some embodiments, the antibody or antigen-binding fragment thereof specifically binds to human CNTFR and cross-reacts with cynomolgus monkey CNTFR. In some embodiments, the antibody or antigen-binding fragment thereof does not cross-react with CNTFR from a non-human species. In some embodiments, the antibody or antigen-binding fragment thereof does not cross-react with mouse CNTFR.

In some embodiments, the anti-CNTFR antibody or antigen-binding fragment thereof comprises:
(a) a heavy chain complementarity determining region 1 (HCDR1) comprising an amino acid sequence selected from the group consisting of SEQ ID NOs: 4 and 24;
(b) a heavy chain complementarity determining region 2 (HCDR2) comprising an amino acid sequence selected from the group consisting of SEQ ID NOs: 6 and 26;
(c) a heavy chain complementarity determining region 3 (HCDR3) comprising an amino acid sequence selected from the group consisting of SEQ ID NOs: 8 and 28;
(d) a light chain complementarity determining region 1 (LCDR1) comprising an amino acid sequence selected from the group consisting of SEQ ID NOs: 12 and 32;
(e) a light chain complementarity determining region 2 (LCDR2) comprising an amino acid sequence selected from the group consisting of SEQ ID NOs: 14 and 34; and
(f) a light chain complementarity determining region 3 (LCDR3) comprising an amino acid sequence selected from the group consisting of SEQ ID NOs: 16 and 36.

In some embodiments, the anti-CNTFR antibody comprises a heavy chain variable region (HCVR) comprising an amino acid sequence that has at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 2 and 22. In some embodiments, the anti-CNTFR antibody comprises an HCVR comprising an amino acid sequence selected from the group consisting of SEQ ID NOs: 2 and 22.

In some embodiments, the anti-CNTFR antibody comprises a light chain variable region (LCVR) comprising an amino acid sequence that has at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs: 10 and 30. In some embodiments, the anti-CNTFR antibody comprises an LCVR comprising an amino acid sequence selected from the group consisting of SEQ ID NOs: 10 and 30.

In some embodiments, the anti-CNTFR antibody or antigen-binding fragment thereof comprises:
(a) an HCDR1 comprising the amino acid sequence of SEQ ID NO:4, an HCDR2 comprising the amino acid sequence of SEQ ID NO:6, and an HCDR3 comprising the amino acid sequence of SEQ ID NO:8; or
(b) an HCDR1 comprising the amino acid sequence of SEQ ID NO:24, an HCDR2 comprising the amino acid sequence of SEQ ID NO:26, and an HCDR3 comprising the amino acid sequence of SEQ ID NO:28.

In some embodiments, the anti-CNTFR antibody comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:2. In some embodiments, the anti-CNTFR antibody comprises an HCVR comprising the amino acid sequence of SEQ ID NO:2. In some embodiments, the anti-CNTFR antibody comprises an HCVR comprising an HCDR1 comprising the amino acid sequence of SEQ ID NO:4, an HCDR2 comprising the amino acid sequence of SEQ ID NO:6, and an HCDR3 comprising the amino acid sequence of SEQ ID NO:8, wherein the HCVR has at least 85%, at least 90%, at least 91%, at least 92%, at least 93%, at least 94%, at least 95%, at least 96%, at least 97%, at least 98%, or at least 99% sequence identity to SEQ ID NO:2. In some embodiments, the anti-CNTFR antibody comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:18. In some embodiments, the anti-CNTFR antibody comprises a heavy chain comprising the amino acid sequence of SEQ ID NO:18.

In some embodiments, the anti-CNTFR antibody comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:22. In some embodiments, the anti-CNTFR antibody comprises an HCVR comprising the amino acid sequence of SEQ ID NO:22. In some embodiments, the anti-CNTFR antibody comprises an HCVR comprising an HCDR1 comprising the amino acid sequence of SEQ ID NO:24, an HCDR2 comprising the amino acid sequence of SEQ ID NO:26, and an HCDR3 comprising the amino acid sequence of SEQ ID NO:28, wherein the HCVR has at least 85%, at least 90%, at least 91%, at least 92%, at least 93%, at least 94%, at least 95%, at least 96%, at least 97%, at least 98%, or at least 99% sequence identity to SEQ ID NO:22. In some embodiments, the anti-CNTFR antibody comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:38. In some embodiments, the anti-CNTFR antibody comprises a heavy chain comprising the amino acid sequence of SEQ ID NO:38.

In some embodiments, the anti-CNTFR antibody or antigen-binding fragment thereof comprises:
(a) an LCDR1 comprising the amino acid sequence of SEQ ID NO:12, an LCDR2 comprising the amino acid sequence of SEQ ID NO:14, and an LCDR3 comprising the amino acid sequence of SEQ ID NO:16; or
(b) an LCDR1 comprising the amino acid sequence of SEQ ID NO:32, an LCDR2 comprising the amino acid sequence of SEQ ID NO:34, and an LCDR3 comprising the amino acid sequence of SEQ ID NO:36.

In some embodiments, the anti-CNTFR antibody comprises an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:10. In some embodiments, the anti-CNTFR antibody comprises an LCVR comprising the amino acid sequence of SEQ ID NO:10. In some embodiments, the anti-CNTFR antibody comprises an LCVR comprising an LCDR1 comprising the amino acid sequence of SEQ ID NO:12, an LCDR2 comprising the amino acid sequence of SEQ ID NO:14, and an LCDR3 comprising the amino acid sequence of SEQ ID NO:16, wherein the LCVR has at least 85%, at least 90%, at least 91%, at least 92%, at least 93%, at least 94%, at least 95%, at least 96%, at least 97%, at least 98%, or at least 99% sequence identity to SEQ ID NO:10. In some embodiments, the anti-CNTFR antibody comprises a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:20. In some embodiments, the anti-CNTFR antibody comprises a light chain comprising the amino acid sequence of SEQ ID NO:20.

In some embodiments, the anti-CNTFR antibody comprises an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:30. In some embodiments, the anti-CNTFR antibody comprises an LCVR comprising the amino acid sequence of SEQ ID NO:30. In some embodiments, the anti-CNTFR antibody comprises an LCVR comprising an LCDR1 comprising the amino acid sequence of SEQ ID NO:32, an LCDR2 comprising the amino acid sequence of SEQ ID NO:34, and an LCDR3 comprising the amino acid sequence of SEQ ID NO:36, wherein the LCVR has at least 85%, at least 90%, at least 91%, at least 92%, at least 93%, at least 94%, at least 95%, at least 96%, at least 97%, at least 98%, or at least 99% sequence identity to SEQ ID NO:30. In some embodiments, the anti-CNTFR antibody comprises a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:40. In some embodiments, the anti-CNTFR antibody comprises a light chain comprising the amino acid sequence of SEQ ID NO:40.

In some embodiments, the anti-CNTFR antibody comprises an HCDR1 comprising the amino acid sequence of SEQ ID NO:4, an HCDR2 comprising the amino acid sequence of SEQ ID NO: 6, an HCDR3 comprising the amino acid sequence of SEQ ID NO:8, an LCDR1 comprising the amino acid sequence of SEQ ID NO:12, an LCDR2 comprising the amino acid sequence of SEQ ID NO:14, and an LCDR3 comprising the amino acid sequence of SEQ ID NO: 16. In some embodiments, the anti-CNTFR antibody comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:2 and an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:10. In some embodiments, the anti-CNTFR antibody comprises an HCVR comprising the amino acid sequence of SEQ ID NO:2 and an LCVR comprising the amino acid sequence of SEQ ID NO: 10. In some embodiments, the anti-CNTFR antibody comprises a heavy chain comprising the amino acid sequence of SEQ ID NO:18 and a light chain comprising the amino acid sequence of SEQ ID NO:20.

In some embodiments, the anti-CNTFR antibody comprises an HCDR1 comprising the amino acid sequence of SEQ ID NO:24, an HCDR2 comprising the amino acid sequence of SEQ ID NO:26, an HCDR3 comprising the amino acid sequence of SEQ ID NO:28, an LCDR1 comprising the amino acid sequence of SEQ ID NO:32, an LCDR2 comprising the amino acid sequence of SEQ ID NO:34, and an LCDR3 comprising the amino acid sequence of SEQ ID NO: 36. In some embodiments, the anti-CNTFR antibody comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:22 and an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:30. In some embodiments, the anti-CNTFR antibody comprises an HCVR comprising the amino acid sequence of SEQ ID NO:22 and an LCVR comprising the amino acid sequence of SEQ ID NO: 30. In some embodiments, the anti-CNTFR antibody comprises a heavy chain comprising the amino acid sequence of SEQ ID NO:38 and a light chain comprising the amino acid sequence of SEQ ID NO:40.

In some embodiments, antigen-binding fragments comprising one or more CDR, HCVR, and/or LCVR sequences disclosed herein (e.g., as disclosed in Table 1, Table 2, or Table 14) are provided.

In some embodiments, the anti-CNTFR antibodies or antigen-binding fragments of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second binding specificity.

### Sequence Variants

The antibodies or antigen-binding fragments of the present disclosure may comprise one or more amino acid substitutions, insertions, and/or deletions in the framework and/or CDR regions of the heavy and/or light chain variable domains as compared to the corresponding germline sequences from which the individual antibodies were derived. Such mutations can be readily ascertained by comparing the amino acid sequences disclosed herein to germ line sequences available from, for example, public antibody sequence databases. The antibodies of the present disclosure may comprise antigen binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antibody was originally derived). Furthermore, the antibodies or antigen-binding fragments may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antibodies or antigen-binding fragments that contain one or more germline mutations can be easily tested for one or more desired property such as, improved binding specificity, increased binding affinity, improved or enhanced antagonistic or agonistic biological properties, reduced immunogenicity, etc. Antibodies and antigen-binding fragments obtained in this general manner are encompassed within the present disclosure.

The present disclosure also includes antibodies or antigen-binding fragments that comprise variants of any of the HCVR, LCVR, and/or CDR amino acid sequences disclosed herein having one or more conservative substitutions. For example, the present disclosure includes antibodies or antigen-binding fragments comprising HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitution(s) relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445. A “moderately conservative” replacement is any change having a nonnegative value in the PAM250 log-likelihood matrix.

The present disclosure also includes antibodies or antigen-binding fragments comprising an HCVR, LCVR, and/or CDR amino acid sequence that is substantially identical to any of the HCVR, LCVR, and/or CDR amino acid sequences disclosed herein. In some embodiments, an antigen-binding molecule comprises HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1 or Table 14. In some embodiments, an antigen-binding molecule comprises HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1 or Table 14, wherein the differences in the amino acid residue(s) relative to the sequence disclosed in Table 1 or Table 14 are conservative substitutions or moderately conservative substitutions.

In some embodiments, the antibody or antigen-binding fragment thereof is chimeric, humanized, or fully human. In some embodiments, the antibody or antigen-binding fragment thereof is humanized. In some embodiments, the antibody or antigen-binding fragment thereof is fully human.

### Polynucleotides, Vectors, and Host Cells

In another aspect, the present disclosure provides nucleic acid molecules comprising one or more polynucleotide sequences encoding the antibodies or antigen-binding fragments disclosed herein, as well as vectors (e.g., expression vectors) encoding such polynucleotide sequences and host cells into which such vectors have been introduced.

In some embodiments, the nucleic acid molecule comprises one or more polynucleotide sequences encoding an antibody or antigen-binding fragment disclosed in Table 1 or Table 14. In some embodiments, the nucleic acid molecule comprises one or more polynucleotide sequences set forth in Table 2 or Table 14.

In some embodiments, the nucleic acid molecule comprises a polynucleotide sequence that encodes an HCVR comprising the HCDR1, HCDR2, and HCDR3 of SEQ ID NOs: 4, 6, and 8, respectively, or of SEQ ID NOs: 24, 26, and 28, respectively. In some embodiments, the nucleic acid molecule comprises a polynucleotide sequence that encodes an HCVR comprising the sequence of SEQ ID NO:2 or 22. In some embodiments, the nucleic acid molecule comprises the polynucleotide sequences of SEQ ID NOs: 3, 5, and 7 or of SEQ ID NOs: 23, 25, and 27. In some embodiments, the nucleic acid molecule comprises the polynucleotide sequence of SEQ ID NO:1 or 21 or has at least 90% sequence identity (e.g., at least 91%, at least 92%, at least 93%, at least 94%, at least 95%, at least 96%, at least 97%, at least 98%, or at least 99% sequence identity) to SEQ ID NO:1 or 21. In some embodiments, the nucleic acid molecule comprises the polynucleotide sequence of SEQ ID NO:17 or 37 or has at least 90% sequence identity (e.g., at least 91%, at least 92%, at least 93%, at least 94%, at least 95%, at least 96%, at least 97%, at least 98%, or at least 99% sequence identity) to SEQ ID NO:17 or 37.

In some embodiments, the nucleic acid molecule comprises a polynucleotide sequence that encodes an LCVR comprising the LCDR1, LCDR2, and LCDR3 of SEQ ID NOs: 12, 14, and 16, respectively, or of SEQ ID NOs: 32, 34, and 36, respectively. In some embodiments, the nucleic acid molecule comprises a polynucleotide sequence that encodes an LCVR comprising the sequence of SEQ ID NO:10 or 30. In some embodiments, the nucleic acid molecule comprises the polynucleotide sequences of SEQ ID NOs: 11, 13, and 15 or of SEQ ID NOs: 31, 33, and 35. In some embodiments, the nucleic acid molecule comprises the polynucleotide sequence of SEQ ID NO:9 or 29 or has at least 90% sequence identity (e.g., at least 91%, at least 92%, at least 93%, at least 94%, at least 95%, at least 96%, at least 97%, at least 98%, or at least 99% sequence identity) to SEQ ID NO:9 or 29. In some embodiments, the nucleic acid molecule comprises the polynucleotide sequence of SEQ ID NO:19 or 39 or has at least 90% sequence identity (e.g., at least 91%, at least 92%, at least 93%, at least 94%, at least 95%, at least 96%, at least 97%, at least 98%, or at least 99% sequence identity) to SEQ ID NO:19 or 39.

In some embodiments, compositions are provided comprising one or more nucleic acid molecules as disclosed herein (e.g., a first nucleic acid molecule comprising a polynucleotide sequence encoding an HCVR of an anti-CNTFR antibody, and a second nucleic acid molecule comprising a polynucleotide sequence encoding an LCVR of an anti-CNTFR antibody).

The present disclosure also provides recombinant expression vectors carrying one or more nucleic acid molecules as disclosed herein, as well as host cells into which such vectors have been introduced. In some embodiments, two or more expression vectors are provided (e.g., a first expression vector comprising a first nucleic acid molecule comprising a polynucleotide sequence encoding an HCVR of an anti-CNTFR antibody, and a second expression vector comprising a second nucleic acid molecule comprising a polynucleotide sequence encoding an LCVR of an anti-CNTFR antibody). In some embodiments, an expression vector comprises two or more nucleic acid molecules (e.g., an expression vector comprising (i) a first nucleic acid molecule comprising a polynucleotide sequence encoding an HCVR of an anti-CNTFR antibody and (ii) a second nucleic acid molecule comprising a polynucleotide sequence encoding an LCVR of an anti-CNTFR antibody). Also provided herein are methods of producing anti-CNTFR antibodies or antigen-binding fragments using the nucleic acid sequences and/or vectors as described herein. In some embodiments, the method of producing the antibody or antigen-binding fragment thereof comprises culturing a host cell comprising one or more nucleic acid sequences and/or expression vectors as described herein under conditions permitting production of the antibodies or antigen-binding fragments, and recovering the antibodies or antigen-binding fragments so produced.

In some embodiments, a host cell comprising one or more nucleic acid sequences and/or vectors as described herein is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing anti-CNTFR antibodies or antigen-binding fragments by culturing the host cells under conditions permitting production of the antibodies or antigen-binding fragments, and recovering the antibodies or antigen-binding fragments so produced.

### Characterization of Anti-CNTFR Antibodies

In some embodiments, the present disclosure includes antibodies and antigen-binding fragments thereof that bind CNTFR (e.g., human CNTFR) with high affinity. In some embodiments, the present disclosure includes antibodies and antigen-binding fragments thereof that bind human CNTFR (e.g., at 25° C. or at 37° C.) with a Ko of less than about 50 nM as measured by surface plasmon resonance or bio-layer interferometry, e.g., using an assay format as defined in Example 2 herein. In some embodiments, the human CNTFR is a monomeric form. In some embodiments, the human CNTFR is a dimeric form. In certain embodiments, the antibodies or antigen-binding fragments of the present disclosure bind human CNTFR with a K_{D} of less than about 50 nM, less than about 40 nM, less than about 30 nM, less than about 20 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured by surface plasmon resonance e.g., using an assay format as defined in Example 2 herein, or a substantially similar assay.

In some embodiments, the present disclosure includes antibodies and antigen-binding fragments thereof that bind human CNTFR (e.g., a monomeric form or a dimeric form of CNTFR) with a dissociative half-life (t½) of greater than about 15 minutes as measured by surface plasmon resonance or bio-layer interferometry at 25° C. or at 37° C., e.g., using an assay format as defined in Example 2 herein, or a substantially similar assay. In certain embodiments, the antibodies or antigen-binding fragments of the present disclosure bind human CNTFR with a t½ of greater than about 15 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 150 minutes, greater than about 200 minutes, greater than about 250 minutes, or greater than about 300 minutes, as measured by surface plasmon resonance at 25° C. or 37° C., e.g., using an assay format as defined in Example 2 herein, or a substantially similar assay.

### Epitope Mapping and Related Technologies

In some embodiments, the epitope on CNTFR to which the antibodies or antigen-binding fragments thereof of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a CNTFR protein. Alternatively, the epitope may consist of a plurality of non-contiguous amino acids (or amino acid sequences) of CNTFR. In some embodiments, the antibodies or antigen-binding fragments of the disclosure may interact with amino acids contained within a CNTFR monomer, or may interact with amino acids on two different chains of a CNTFR dimer, or may interact with amino acids of CNTFR as part of a complex with CNTFR, gp130, and LIFR. The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antibody “interacts with one or more amino acids” within a polypeptide or protein. Exemplary techniques that can be used to determine an epitope or binding domain of a particular antibody include, e.g., routine crossblocking assay such as that described in Antibodies, Harlow and Lane (Cold Spring Harbor Press, Cold Spring Harbor, NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73: 256A-265A. X-ray crystal structure analysis can also be used to identify the amino acids within a polypeptide with which an antibody interacts.

In some embodiments, the present disclosure provides anti-CNTFR antibodies that bind to the same epitope as any of the specific exemplary antibodies described herein (e.g., antibodies comprising any of the amino acid sequences set forth in Table 1 or Table 14 below). In some embodiments, the anti-CNTFR antibody binds to an epitope in D1 domain. In some embodiments, the anti-CNTFR antibody binds to an epitope within amino acids Q23-V105 of CNTFR. In some embodiments, the anti-CNTFR antibody binds to an epitope in D2 or D3 domain. In some embodiments, the anti-CNTFR antibody binds to an epitope within amino acids V105-D205 of CNTFR.

In some embodiments, the present disclosure provides anti-CNTFR antibodies that compete for binding to CNTFR with any of the specific exemplary anti-CNTFR antibodies described herein.

One skilled in the art can determine whether a particular antibody thereof binds to the same epitope as, or competes for binding with, a reference antibody of the present disclosure by using routine methods known in the art. For example, to determine if a test antibody binds to the same epitope on CNTFR as a reference antibody of the present disclosure, the reference antibody is first allowed to bind to a CNTFR protein. Next, the ability of a test antibody to bind to the CNTFR protein is assessed. If the test antibody is able to bind to CNTFR following saturation binding with the reference antibody, it can be concluded that the test antibody binds to a different epitope of CNTFR than the reference antibody. On the other hand, if the test antibody is not able to bind to CNTFR following saturation binding with the reference antibody, then the test antibody may bind to the same epitope of CNTFR as the epitope bound by the reference antibody of the disclosure. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antibodies bind to the same (or overlapping) epitope if, e.g., a 1-, 2-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if a test antibody or antigen-binding fragment thereof competes for binding with a reference antibody, the above-described binding methodology is performed in two orientations: In a first orientation, the reference antibody is allowed to bind to a CNTFR protein under saturating conditions followed by assessment of binding of the test antibody to the CNTFR protein. In a second orientation, the test antibody is allowed to bind to a CNTFR protein under saturating conditions followed by assessment of binding of the reference antibody to the CNTFR protein. If, in both orientations, only the first (saturating) antibody is capable of binding to CNTFR, then it is concluded that the test antibody and the reference antibody compete for binding to CNTFR. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Preparation of Antibodies

Antibodies as disclosed herein can be prepared by any antibody generating technology known in the art. In certain embodiments, one or more of the individual components (e.g., heavy and light chains) of the antibodies are derived from chimeric, humanized or fully human antibodies. Methods for making such antibodies are well known in the art. For example, one or more of the heavy and/or light chains of the antibodies of the present disclosure can be prepared using VELOCIMMUNE™ technology. Using VELOCIMMUNE™ technology (or any other human antibody generating technology), high affinity chimeric antibodies to a particular antigen (e.g., CNTFR) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the antibodies.

In some embodiments, antibodies can be generated by administering to a mouse an immunogen (e.g., a peptide as shown in SEQ ID NO:42, optionally conjugated to a carrier), optionally in combination with an adjuvant. Following immunization, splenocytes are harvested and fused with mouse myeloma cells to preserve their viability and form hybridoma cell lines. The hybridoma cell lines are screened and selected to identify cell lines that produce CNTFR-specific antibodies. Alternatively, DNA encoding antigen-specific chimeric antibodies or the variable domains of the light and heavy chains can be isolated directly from antigen-positive B cells without fusion to myeloma cells, as described in US 2007/0280945 or WO 2016/077666, each of which is incorporated by reference in its entirety.

In some embodiments, genetically engineered animals may be used to make human antibodies. Non-limiting exemplary genetically modified mice, and methods of generating antibodies from genetically modified mice, are described in U.S. Pat. Nos. 8,697,940, 10,130,081, 10,561,124, and 10,640,800, the entire contents of each of which are incorporated by reference herein. As used herein, “fully human” refers to an antigen-binding molecule, e.g., an antibody, or antigen-binding fragment or immunoglobulin domain thereof, comprising an amino acid sequence encoded by a DNA derived from a human sequence over the entire length of each polypeptide of the antigen-binding molecule, antibody, antigen-binding fragment, or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### Bioequivalents

The present disclosure encompasses antibodies having amino acid sequences that vary from those of the described antibodies but that retain the ability to bind CNTFR. Such variant molecules comprise one or more additions, deletions, or substitutions of amino acids when compared to a parent sequence, but exhibit biological activity that is essentially equivalent to that of the described antibodies. Likewise, the nucleic acid sequences encoding the antibodies of the present disclosure encompass sequences that comprise one or more additions, deletions, or substitutions of nucleotides when compared to the disclosed sequence, but that encode an antibody that is essentially bioequivalent to the antibodies disclosed herein.

The present disclosure includes antibodies that are bioequivalent to any of the exemplary antibodies set forth herein. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the first antibody (e.g., reference product) and the second antibody (e.g., biological product) without an expected increase in the risk of adverse effects, including a clinically significant change in immunogenicity, or diminished effectiveness, as compared to continued therapy without such switching.

In one embodiment, two antibodies are bioequivalent if they both act by a common mechanism or mechanisms of action for the condition or conditions of use, to the extent that such mechanisms are known.

Bioequivalence may be demonstrated by in vivo and in vitro methods. Non-limiting examples of bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary antibodies set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other embodiments, bioequivalent antibodies may include the exemplary antibodies set forth herein comprising amino acid changes which modify the glycosylation characteristics of the antibodies, e.g., mutations which eliminate or remove glycosylation.

### Pharmaceutical Compositions

In another aspect, the present disclosure provides pharmaceutical compositions comprising the anti-CNTFR antibodies and antigen-binding fragments disclosed herein. The pharmaceutical compositions are formulated with one or more pharmaceutically acceptable vehicles, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a patient according to the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions as disclosed herein may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, an antibody or antigen-binding fragment as disclosed herein, or a pharmaceutical composition comprising an antibody or antigen-binding fragment as disclosed herein is contained within a container. Thus, in another aspect, containers comprising an antibody or pharmaceutical composition as disclosed herein are provided. For example, in some embodiments, an antibody or pharmaceutical composition is contained within a container selected from the group consisting of a glass vial, a syringe, a pen delivery device, and an autoinjector.

In some embodiments, an antibody or pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver an antibody or pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park Ill.).

In some embodiments, the antibody or pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc. In some embodiments, the amount of the antigen-binding molecule contained in the dosage form is about 5 to about 500 mg, e.g., from about 5 to about 100 mg or from about 10 to about 250 mg.

### Therapeutic Uses

In another aspect, the present disclosure provides for methods of using the anti-CNTFR antibodies and antigen-binding fragments disclosed herein. In some embodiments, the anti-CNTFR antibodies and antigen-binding fragments disclosed herein are useful for the treatment, prevention and/or amelioration of any disease, disorder, or condition associated with CNTFR expression or activity.

In some embodiments, the disease, disorder, or condition is a metabolic disease. Non-limiting examples of metabolic diseases include obesity, dyslipidemia, hyperglycemia, hyperlipidemia, hypercholesterolemia, hypertriglyceridemia, hyperinsulinemia, and/or hypertension, type 1 diabetes, type 2 diabetes, insulin resistance, obesity-induced insulin resistance, metabolic syndrome, diet-induced food craving, diabetic nephropathy, diabetic neuropathy, and diabetic ocular complications (e.g., retinopathy, cataract formation, or glaucoma).

In some embodiments, the disease, disorder, or condition is a neurological or neurodegenerative disease. Non-limiting examples of neurological or neurodegenerative diseases include Alzheimer's disease, amyotrophic lateral sclerosis, ataxia, demyelinating disease, Huntington's disease, Lewy body disease, motor neuron disease, multiple sclerosis, multiple system atrophy, progressive supranuclear palsy, retinal degenerative diseases (e.g., retinitis pigmentosa, macular degeneration, age-related macular degeneration, or Stargardt disease), Parkinson's disease, and spinal muscular atrophy.

### Dosage and Administration Regimens

In some embodiments, an amount of an anti-CNTFR antibody or antigen-binding fragment that is administered to a subject according to the methods disclosed herein is a therapeutically effective amount. As used herein, the phrase “therapeutically effective amount” means an amount that produces the desired effect for which it is administered.

In some embodiments, the antibody is administered to a subject as a weight-based dose. A “weight-based dose” (e.g., a dose in mg/kg) is a dose of the antibody that will change depending on the subject's weight.

In other embodiments, the antibody is administered as a fixed dose. A “fixed dose” (e.g., a dose in mg) means that one dose of the antibody is used for all subjects regardless of any specific subject-related factors, such as weight. In one particular embodiment, a fixed dose of an antibody is based on a predetermined weight or age.

Typically, a suitable dose of the antibody can be in the range of about 0.001 to about 200.0 milligram per kilogram body weight of the recipient, generally in the range of about 1 to 50 mg per kilogram body weight. For example, the antibody can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg per single dose. Values and ranges intermediate to the recited values are also intended to be part of this disclosure.

In some embodiments, the antibody is administered as a fixed dose of between about 0.05 mg to about 2500 mg, e.g., from about 0.05 mg to about 1000 mg, from about 0.1 mg to about 500 mg, from about 0.1 mg to about 100 mg, from about 50 mg to about 2000 mg, or from about 50 mg to about 1000 mg. In some embodiments, the antibody is administered as a fixed dose of about 0.05 mg, about 0.1 mg, about 0.5 mg, about 1 mg, about 5 mg, about 10 mg, about 15 mg, about 20 mg, about 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

In some embodiments, the antibody is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, multiple doses of an antibody as disclosed herein are administered to a subject over a defined time course. In some embodiments, the methods of the present disclosure comprise sequentially administering to a subject multiple doses of the antibody. As used herein, “sequentially administering” means that each dose of the antibody is administered to the subject at a different point in time, e.g., on different days separated by a predetermined interval (e.g., hours, days, weeks, or months). In some embodiments, the methods of the disclosure comprise sequentially administering to the patient a single initial dose of the antibody, followed by one or more secondary doses of the antibody, and optionally followed by one or more tertiary doses of the antibody.

The terms “initial dose,” “secondary dose(s),” and “tertiary dose(s)” refer to the temporal sequence of administration of the antibody. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. In some embodiments, the initial, secondary, and tertiary doses may all contain the same amount of the antibody, but may differ from one another in terms of frequency of administration. In some embodiments, the amount of the antibody contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial dose and the one or more secondary doses each contain the same amount of the antibody. In other embodiments, the initial dose comprises a first amount of the antibody, and the one or more secondary doses each comprise a second amount of the antibody. For example, the first amount of the antibody can be 1.5×, 2×, 2.5×, 3×, 3.5×, 4× or 5× or more than the second amount of the antibody.

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of the antibody that is administered to a patient prior to the administration of the very next dose in the sequence with no intervening doses.

The methods of the disclosure may comprise administering to a patient any number of secondary and/or tertiary doses of an antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1, 2, 3, or 4 weeks after the immediately preceding dose. Similarly, in some embodiments involving multiple tertiary doses, each tertiary dose is administered at the same frequency as the other tertiary doses. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Combination Therapies

In some embodiments, an antibody or antigen-binding fragment of the disclosure can be used in combination with one more other therapeutic agents. In some embodiments, the additional therapeutic agent(s) is an antibody, small molecule, inhibitory nucleic acid (e.g., RNAi), antibody drug conjugate, bispecific antibody, or a combination thereof.

In some embodiments, an anti-CNTFR antibody of the disclosure can be administered in combination with CNTF, a modified or mutated version of CNTF, or a CNTF derivative.

In some embodiments, an anti-CNTFR antibody of the disclosure can be administered in combination with one or more therapeutic agents for the treatment of a metabolic disease, e.g., obesity, hyperglycemia, hyperlipidemia, type 2 diabetes, type 1 diabetes, appetite control, etc. In some embodiments, an anti-CNTFR antibody of the disclosure can be administered in combination with one or more therapeutic agents for the treatment of a neurological or neurodegenerative disease.

The additional therapeutically active component(s) may be administered just prior to, concurrent with, or shortly after the administration of an antibody of the present disclosure. For the purposes of the present disclosure, such administration regimens are considered the administration of an antibody “in combination with” an additional therapeutically active component.

The present disclosure includes pharmaceutical compositions in which an antibody of the present disclosure is co-formulated with one or more of the additional therapeutically active component(s) as described elsewhere herein.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Human Antibodies to Human CNTFR

Antibodies against human CNTFR were obtained as follows: Single Rearranged Human Light Chain mouse strains (Universal light chain mice; ULC) expressing either Vκ 3-20Jκ 1 or Vκ 1-39Jκ 5 light chains (see, e.g., WO 2011/097603) were initially immunized via footpad with human CNTFR expressed with a C-terminal mFc tag (SEQ ID NO:45), which was then switched to either human CNTFR expressed with a C-terminal hFc tag (SEQ ID NO:46) or human CNTFR derived from E. coli production. Antigen-positive B cells were sorted (e.g., as described in U.S. Pat. No. 7,582,298, incorporated by reference herein) using biotinylated human CNTFR expressed with a C-terminal myc-myc-hexahistidine tag to obtain fully human anti-CNTFR antibodies.

Anti-CNTFR antibodies generated using this method include the antibodies designated REGN8938 and mAb25311. Certain biological properties of the exemplary anti-CNTFR antibodies generated in accordance with the methods of this Example are described in detail in the Examples set forth below.

TABLE 1
Amino Acid Sequence Identifiers

| Antibody | SEQ ID NOS: | | | | | | | | | |
|---|---|---|---|---|---|---|---|---|---|---|
| Designation | HCVR | HCDR1 | HCDR2 | HCDR3 | HC | LCVR | LCDR1 | LCDR2 | LCDR3 | LC |
| REGN8938 | 2 | 4 | 6 | 8 | 18 | 10 | 12 | 14 | 16 | 20 |
| mAb25311 | 22 | 24 | 26 | 28 | 38 | 30 | 32 | 34 | 36 | 40 |

TABLE 2
Nucleic Acid Sequence Identifiers

| Antibody | SEQ ID NOS: | | | | | | | | | |
|---|---|---|---|---|---|---|---|---|---|---|
| Designation | HCVR | HCDR1 | HCDR2 | HCDR3 | HC | LCVR | LCDR1 | LCDR2 | LCDR3 | LC |
| REGN8938 | 1 | 3 | 5 | 7 | 17 | 9 | 11 | 13 | 15 | 19 |
| mAb25311 | 21 | 23 | 25 | 27 | 37 | 29 | 31 | 33 | 35 | 39 |

### Example 2: Binding Kinetics of Anti-CNTFR Antibodies

Equilibrium dissociation constants (KD) for CNTFR binding to purified anti-CNTFR monoclonal antibodies were determined using a real-time surface plasmon resonance (SPR) based Biacore 4000 biosensor. All binding studies were performed in 10 mM HEPES, 150 mM NaCl, 3 mM EDTA, and 0.05% v/v surfactant Tween-20, pH 7.4 (HBS-ET) running buffer at 25° C. and 37° C. The Biacore CM4 sensor surface was first derivatized by amine coupling with a monoclonal mouse anti-human Fc antibody (REGN2567) to capture anti-CNTFR monoclonal antibodies. Different concentrations of CNTFR reagents, human CNTFR extracellular domain expressed with a C-terminal myc-myc-hexahistidine tag (hCNTFR-mmh; SEQ ID NO:42), monkey CNTFR extracellular domain expressed with a C-terminal myc-myc-hexahistidine tag (mfCNTFR-mmh; SEQ ID NO:43), mouse CNTFR extracellular domain expressed with a C-terminal myc-myc-hexahistidine tag (mCNTFR-mmh; SEQ ID NO:44), and human CNTFR extracellular domain expressed with a C-terminal mouse IgG2a Fc tag (hCNTFR-mFc; SEQ ID NO: 45), at concentrations ranging from 6.25 nM to 100 nM in a series of 4-fold dilutions prepared in HBS-ET running buffer were injected at a flow rate of 30 μL/min for 2.5 minutes. The dissociation of different CNTFR reagents bound to anti-CNTFR monoclonal antibodies was monitored for 10 minutes in HBS-ET running buffer. At the end of each cycle, the anti-CNTFR monoclonal antibodies capture surface was regenerated using a 12 sec injection of 20 mM H3PO4. The association rate (ka) and dissociation rate (kd) were determined by fitting the real-time specific binding sensorgrams to a 1:1 binding model with mass transport limitation using Scrubber 2.0c curve-fitting software. Binding dissociation equilibrium constant (KD) and dissociative half-life (t½) were calculated from the kinetic rates as:

$${K}_{D} ( M ) = \frac{k d}{k a} , and t \frac{1}{2} ( min ) = \frac{ln ( 2 )}{60 * k d}$$

Binding kinetics parameters for different CNTFR reagents to anti-CNTFR monoclonal antibodies of the disclosure at 25° C. and 37° C. are shown in Table 3 through Table 10.

At 25° C., anti-CNTFR monoclonal antibody REGN8938 bound to monomeric hCNTFR-mmh with KD value of 14.2 nM, while mAb25311P2 bound weakly with small binding signal too low for KD determination (Table 3). At 37° C., anti-CNTFR monoclonal antibody REGN8938 bound to monomeric hCNTFR-mmh with KD value of 45.1 nM, while mAb25311P2 binding signal was too low for KD determination (Table 4).

At 25° C., anti-CNTFR monoclonal antibodies REGN8938 and mAb25311P2 bound to dimeric hCNTFR-mFc with KD values of 45.1 μM and 7.22 nM respectively, as shown in Table 5. At 37° C., anti-CNTFR monoclonal antibodies REGN8938 and mAb25311P2 bound to dimeric hCNTFR-mFc with KD values of 71.7 pM and 27.2 nM respectively, as shown in Table 6.

At 25° C., anti-CNTFR monoclonal antibody REGN8938 bound to monomeric mfCNTFR-mmh with KD value of 12.4 nM, while mAb25311P2 bound weakly with small binding signal too low for KD determination (Table 7). At 37° C., anti-CNTFR monoclonal antibody REGN8938 bound to monomeric hCNTFR-mmh with KD value of 41.9 nM, while mAb25311P2 binding signal was too low for KD determination (Table 8).

At both 25° C. and 37° C., anti-CNTFR monoclonal antibodies REGN8938 and mAb25311P2 showed no binding to mCNTFR-mmh, as shown in Table 9 and Table 10, respectively.

TABLE 3
Kinetic binding parameters for the interaction of hCNTFR-mmh with
anti-CNTFR monoclonal antibodies at 25° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 302 ± 0.6 | 89 | 1.15E+05 | 1.63E−03 | 1.42E−08 | 7 |
| mAb25311P2 | 242 ± 0.8 | 4 | IC | IC | IC | IC |

IC indicates that the binding data was inconclusive

TABLE 4
Kinetic binding parameters for the interaction of hCNTFR-mmh with
anti-CNTFR monoclonal antibodies at 37° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 346 ± 0.9 | 93 | 1.63E+05 | 7.37E−03 | 4.51E−08 | 1.6 |
| mAb25311P2 | 312 ± 0.8 | 6 | IC | IC | IC | IC |

IC indicates that the binding data was inconclusive

TABLE 5
Kinetic binding parameters for the interaction of hCNTFR-mFc with anti-CNTFR
monoclonal antibodies at 25° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 302 ± 0.5 | 158 | 3.10E+05 | 1.45E−05 | 4.51E−11 | 798 |
| mAb25311P2 | 242 ± 0.5 | 13 | 1.73E+04 | 1.25E−04 | 7.22E−09 | 93 |

TABLE 6
Kinetic binding parameters for the interaction of hCNTFR-mFc with anti-CNTFR
monoclonal antibodies at 37° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 344 ± 0.9 | 204 | 4.25E+05 | 3.04E−05 | 7.17E−11 | 379 |
| mAb25311P2 | 309 ± 0.7 | 26 | 2.88E+04 | 7.84E−04 | 2.72E−08 | 15 |

TABLE 7
Kinetic binding parameters for the interaction of mfCNTFR-mmh with anti-CNTFR
monoclonal antibodies at 25° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 303 ± 0.3 | 93 | 1.21E+05 | 1.51E−03 | 1.24E−08 | 8 |
| mAb25311P2 | 242 ± 0.8 | 4 | IC | IC | IC | IC |

IC indicates that the binding data was inconclusive

TABLE 8
Kinetic binding parameters for the interaction of mfCNTFR-mmh with anti-CNTFR
monoclonal antibodies at 37° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 345 ± 0.9 | 96 | 1.67E+05 | 6.99E−03 | 4.19E−08 | 1.7 |
| mAb25311P2 | 311 ± 0.7 | 5 | IC | IC | IC | IC |

IC indicates that the binding data was inconclusive

TABLE 9
Kinetic binding parameters for the interaction of mCNTFR-mmh
with anti-CNTFR monoclonal antibodies at 25° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 302 ± 0.5 | 0 | NB | NB | NB | NB |
| mAb25311P2 | 243 ± 0.1 | 0 | NB | NB | NB | NB |

NB indicates that no binding was observed under the current experimental conditions

TABLE 10
Kinetic binding parameters for the interaction of mCNTFR-mmh
with anti-CNTFR monoclonal antibodies at 37° C.

| | | Ag | | | | |
|---|---|---|---|---|---|---|
| | mAb | Bound | | | | |
| | Capture | at 100 | kₐ | k_{d} | K_{D} | t½ |
| REGN# | Level (RU) | nM (RU) | (1/Ms) | (1/s) | (M) | (min) |
| REGN8938 | 342 ± 0.3 | 0 | NB | NB | NB | NB |
| mAb25311P2 | 309 ± 0.2 | 0 | NB | NB | NB | NB |

NB indicates that no binding was observed under the current experimental conditions

### Example 3: Anti-CNTFR Antibody Binding to Chimeric CNTFR Domain Proteins

Binding of purified anti-CNTFR monoclonal antibodies to human/mouse chimeric CNTFR domain proteins were determined using a real-time surface plasmon resonance (SPR) based Biacore S200 biosensor. All binding studies were performed in 10 mM HEPES, 150 mM NaCl, 3 mM EDTA, and 0.05% v/v surfactant Tween-20, pH 7.4 (HBS-EP) running buffer at 25° C. To prepare myc capturing surfaces, CM5 Biacore sensor surfaces were derivatized by amine coupling with a monoclonal anti-myc antibody (REGN642) to capture human CNTFR or human/mouse chimeric CNTFR domain proteins as follows: human CNTFR extracellular domain (Q23-S342 of accession number P26992) expressed with a C-terminal myc-myc-hexahistidine tag (also referred to herein as “hCNTFR-mmh”; SEQ ID NO:42), human CNTFR extracellular domain (Q23-V105)/mCNTFR (G106-S342) expressed with a C-terminal myc-myc-hexahistidine tag (also referred to herein as “hCNTFR (Q23-V105)/mCNTFR (G106-S342).mmh”; SEQ ID NO:47), and human CNTFR extracellular domain (Q23-D205)/mCNTFR (P206-S342) expressed with a C-terminal myc-myc-hexahistidine tag (also referred to herein as “hCNTFR (Q23-D205)/mCNTFR (P206-S342).mmh”; SEQ ID NO:48).

Anti-CNTFR monoclonal antibodies at concentrations of 20 nM and 100 nM prepared in HBS-EP running buffer were injected at a flow rate of 50 L/min for 4 minutes. The dissociation of different anti-CNTFR monoclonal antibodies bound to human/mouse chimeric CNTFR domain proteins was monitored for 4 minutes in HBS-EP running buffer. At the end of each cycle, the human/mouse chimeric CNTFR domain protein capture surface was regenerated using a 12 sec injection of 20 mM H3PO4. The binding response were determined using Scrubber 2.0c curve-fitting software.

Specific binding responses of anti-CNTFR monoclonal antibodies at a concentration of 100 nM to the surfaces of human/mouse chimeric CNTFR domain proteins were determined at the end of the antibody injections and are presented in Table 11.

mAb25311P2 showed comparable binding signals to the surfaces of hCNTFR-mmh and hCNTFR (Q23-D205)/mCNTFR (P206-S342).mmh, but not hCNTFR (Q23-V105)/mCNTFR (G106-S342).mmh, implying its binding epitope resides in the region of V105-D205 of hCNTFR.

REGN8938 showed comparable binding signals to all three surfaces of hCNTFR-mmh, hCNTFR (Q23-V105)/mCNTFR (G106-S342).mmh, and hCNTFR (Q23-D205)/mCNTFR (P206-S342).mmh, suggesting its binding epitope resides in the region of Q23-V105.

No binding of isotype control mAb (REGN1945) were observed, as expected.

TABLE 11
Determination of the binding ability of anti-CNTFR Monoclonal Antibodies to hCNTF-mmh and human/mouse chimeric
CNTFR domain proteins at 25° C.

| | | | hCNTFR (Q23-V105)_ | | | | |
|---|---|---|---|---|---|---|---|
| | hCNTFR.mmh Surface | | mCNTFR (G106- | | hCNTFR (Q23-D205)_ | | |
| | Capture | | S342).mmh | | mCNTFR(P206-S342).mmh | | CNTFR |
| Injected | Level | 100 nM mAb | Capture | 100 nM mAb | Capture | 100 nM mAb | Binding |
| Antibody | (RU) | Bound (RU) | Level (RU) | Bound (RU) | Level (RU) | Bound (RU) | Domain |
| mAb25311P2 | 145 ± 0.1 | 80 | 152 ± 0.2 | 1 | 148 ± 0.2 | 89 | V105 - D205 |
| REGN8938 | 144 ± 0.3 | 220 | 150 ± 0.2 | 232 | 147 ± 0.1 | 238 | Q23-V105 |
| Isotype control | 139 ± 0.2 | 1 | 146 ± 0.4 | 0 | 143 ± 0.2 | 0 | N/A |

### Example 4: Functional Activity of Anti-hCNTFR Antibodies on Signaling in a Cell-Based Bioassay

CNTFR is a Glycosylphosphatidylinositol (GPI) anchored receptor and a member of the Class I cytokine receptor family. Ligand mediated interaction of CNTFR with gp130 and LIFR leads to signal transduction via Janus family tyrosine kinases (Jaks) and signal transducers and activators of transcription (STATs) (Davis et al., Science 1993, 260:1805-1808; Elson et al., Nat Neurosci 2000, 3:867-872). In order to assess the effects of anti-CNTFR antibodies on signaling, a bioassay was established using IMR-32 cells (human brain neuroblastoma, ATCC CCL-127TM) overexpressing a STAT3-response element fused to firefly luciferase reporter (STAT3-Luc). IMR-32 cells endogenously express CNTFR, gp130, and LIFR, along with all other components of the Jak-STAT signal transduction machinery. The resulting cell line is named IMR-32/STAT3-luc.

For the bioassay, IMR-32/STAT3-luc cells were plated in assay buffer (Optimem+0.1% FBS+Pen/Strep/L-Glutamine) at 15,000 cells/well in a 96-well plate and incubated overnight at 37° C. in 5% CO2. The following day, anti-CNTFR antibodies or a human IgG control antibody were serially diluted 1:4 in assay buffer from 300 nM to 73.2 μM (plus a sample containing assay buffer alone without test molecule) and then added to the cells with either 10 μM human Ciliary Neurotrophic Factor (hCNTF, R&D Systems, 257-NT), 300 PM of the human Cytokine-like Factor 1/human Cardiotrophin-like cytokine complex (hCLF-1/CLC, R&D Systems 1151-CL), or no ligand. To obtain a dose dependent activation by the ligands, both hCNTF and hCLF-1/CLC were serially diluted 1:4 from 10 nM to 2.44 μM in assay buffer (plus a sample containing buffer alone without test molecule) and added to cells without antibodies. After a 5 hour incubation at 37° C. in 5% CO2, luciferase activity was measured using OneGlo™ reagent (Promega, #E6031) and an Envision plate reader (Perkin Elmer). The results were analyzed using nonlinear regression (4-parameter logistics) with Prism software (GraphPad) to obtain EC₅₀ and IC₅₀ values.

The percentage of activation was calculated with the RLU values using the following equation:

$$Maximum Percent activation = \frac{RL {U}_{Exp . Act .} - R L {U}_{Background}}{RL {U}_{Max . Act .} - {RFU}_{Background}} ⨯ 100$$

In this equation “RLU_{Exp. Act.}” are the highest luminescence values recorded across the tested range of antibody concentrations, “RLU_{Max. Act.}” are the highest luminescence values observed from cells treated with various concentrations of CNTF, and “RLU_{Background}” is the luminescence value from cells without ligands or antibodies.

The percentage of inhibition was calculated with the RLU values using the following equation:

$$Maximum Percent inhibition = \frac{RL {U}_{Baseline} - R L {U}_{Exp . Inh .}}{RL {U}_{Baseline} - {RFU}_{Background}} ⨯ 100$$

In this equation “RLU_{Baseline}” is the luminescence value from the cells treated with either 10 PM hCNTF or 300 μM hCLF1/CLC without antibodies, “RLU_{Exp. Inh}” are the lowest luminescence values recorded across the tested range of antibody concentrations, and “RLU_{Background}” is the luminescence value from cells without ligands or antibodies.

The anti-CNTFR antibodies of the disclosure were tested for a functional effect on IMR-32/STAT3-luc cells with or without ligands. As shown in Table 12, none of the antibodies of the invention activated cells without ligands. REGN8938 showed 14.3% inhibition of hCNTF and 21.4% inhibition of hCLF-1/CLC. Due to the small dynamic range of inhibition, the IC₅₀ values could not be conclusively calculated. mAb25311P2 and the human IgG control showed no inhibition of either hCNTF or hCLF-1/CLC. Dose responses of hCNTF and hCLF-1/CLC were shown to activate IMR-32/STAT3-luc cells with EC₅₀ values of 2.26 PM and 96.3 pM, respectively.

TABLE 12
Functional effect of anti-CNTFR antibodies on IMR-32/STAT3-luc cells with or
without ligands

| Ligand | None | | hCNTF | | hCLF-1/CLC | |
|---|---|---|---|---|---|---|
| Ligand EC50 [M] | N/A | | 2.26E−12 | | 9.63E−11 | |
| Constant Ligand | No Ligand | | 10 pM hCNTF | | 300 pM hCLF-1/CLC | |
| Concentration | | | | | | |
| | | | Max. % | | Max. % | |
| Antibody | % Activation | EC₅₀ [M] | Inhibition | IC₅₀ [M] | Inhibition | IC₅₀ [M] |
| mAb25311P2 | No Activation | No Activation | No Inhibition | No Inhibition | No Inhibition | No Inhibition |
| REGN8938 | No Activation | No Activation | 14.3 | NC | 21.4 | NC |
| hIgG Control | No Activation | No Activation | No Inhibition | No Inhibition | No Inhibition | No Inhibition |

NC: IC50 values not calculated due to the small dynamic range of inhibition.

### Example 5: Cell Binding by Flow Cytometry with HEK293/hCNTFR, HEK293/mfCNTFR, and HEK293/mCNTFR Cells

In order to assess cell binding by anti-CNTFR antibodies of the disclosure, HEK293 cells (human embryonic kidney, ATCC #CRL-1573™) were engineered to stably overexpress full length CNTFR, from either human (hCNTFR, amino acids 1-372, UniProtKB/Swiss-Prot Accession #P26992), cynomolgus monkey (mfCNTFR, amino acids 1-372, NCBI Reference Sequence Accession #XP_005581519.1), or mouse (mCNTFR, amino acids 1-372, UniProtKB/Swiss-Prot Accession #088507). All lines were sorted for high expression of CNTFR, and the resulting cell lines are referred to as HEK293/hCNTFR, HEK293/mfCNTFR, and HEK293/mCNTFR.

To assess binding of the anti-CNTFR antibodies to CNTFR receptors expressed on the cell surface, each of the HEK293/CNTFR cells listed above or HEK293 parental cells were diluted to 5×10⁵ cells/well in 96-well v-bottom plates. Antibodies were serially diluted (1:4) with final antibody concentrations ranging from 22.9 pM to 375 nM, added to cells, and incubated at 4° C. for 30 minutes. Cells were then washed and incubated with 4 μg/mL of Alexa Fluor 647-conjugated anti-human secondary antibody (Jackson ImmunoResearch Laboratories Inc., #109-607-003) at 4° C. for 30 minutes. Cells were fixed using BD CytoFix™ (Becton Dickinson, #554655), filtered, and then analyzed on IQuePlus® Flow Cytometer (Intellicyt®). Geometric Mean Fluorescent Intensity (MFI) values were calculated by Forecyt Software and plotted in GraphPad Prism using a 4-parameter logistic equation over the 8-point dose-response curve to obtain EC₅₀ values. The maximal fold change in binding signal over background (Max. Fold Binding) was calculated as the highest MFI observed for antibody binding divided by MFI observed with secondary antibody alone for the corresponding cells. The secondary only condition was excluded from the EC₅₀ calculation.

As shown in Table 13, both antibodies bound to HEK293/hCNTFR and HEK293/mfCNTFR cells with Max. Fold Binding values ranging from 235 to 706 and EC₅₀ values ranging from 1.09 nM to 4.33 nM. REGN8938 bound to HEK293/mCNTFR cells with a Max. Fold Binding value of 49 and an EC₅₀ value of 14 nM and to HEK293 parental cells with a Max. Fold Binding value of 5 and an EC₅₀ value of 240 pM. mAb25311P2 showed no binding to HEK293/mCNTFR or parental HEK293 cells. The human IgG control antibody showed no binding to all cell lines tested.

TABLE 13
Binding of anti-CNTFR antibodies to HEK293/CNTFR cells

| | HEK293 parental | | HEK293/hCNTFR | | HEK293/mfCNTFR | | HEK293/mCNTFR | |
|---|---|---|---|---|---|---|---|---|
| | Max. Fold | | Max. Fold | Max. Fold | Max. Fold | | Max. Fold | |
| Antibody | Binding | EC₅₀ [M] | Binding | Binding | Binding | EC₅₀ [M] | Binding | EC₅₀ [M] |
| mAb25311P2 | NB | NB | 510 | 4.33E−09 | 235 | 2.8E−09 | NB | NB |
| REGN8938 | 5* | 2.4E−10* | 706 | 1.09E−09 | 521 | 1.4E−09 | 49 | 1.4E−08 |
| hIgG Control | NB | NB | NB | NB | 1 | Weak | NB | NB |

NB: no binding detected.
*There were no cells in the 375nM condition for this antibody. The data listed is for the remaining 7 points.

TABLE 14
Informal Sequence Listing

| SEQ | | |
|---|---|---|
| ID NO | Sequence | Description |
| 1 | CAGGTGCAGCTGGTGGAGTCTGGGGGAGACGTGGTCCAGCCTGGGAGGTCCC | REGN8938 HCVR nucleotide |
| | TGAGACTCTCCTGTGCAGCCTCTGGATTCACCTTCAGTTACTATGGCATGCACTG | sequence |
| | GGTCCGCCAGCCTCCAGGCAAGGGGCTGGAGTGGGTGACAATTATATCATTTG | |
| | ATGGAAGTCAAAAATACTATGCAGACTCCGTGAAGGGCCGCTTCACCATCTCCA | |
| | GAGACAATTCCAAGAACACGGTTTTTTTGCAAATGAACAGCCTGAGAACAGAG | |
| | GACACGGGTTTCTATTACTGTGCGGCCCAAAGTAGTACCTGGCCCGAATACTTC | |
| | CAGTACTGGGGCCAGGGCACCCTGGTCACCGTCTCCTCA | |
| 2 | QVQLVESGGDVVQPGRSLRLSCAASGFTFSYYGMHWVRQPPGKGLEWVTIISFDG | REGN8938 HCVR amino acid |
| | SQKYYADSVKGRFTISRDNSKNTVFLQMNSLRTEDTGFYYCAAQSSTWPEYFQYW | sequence |
| | GQGTLVTVSS | |
| 3 | GGATTCACCTTCAGTTACTATGGC | REGN8938 HCDR1 nucleotide |
| | | sequence |
| 4 | GFTFSYYG | REGN8938 HCDR1 amino acid |
| | | sequence |
| 5 | ATATCATTTGATGGAAGTCAAAAA | REGN8938 HCDR2 nucleotide |
| | | sequence |
| 6 | ISFDGSQK | REGN8938 HCDR2 amino acid |
| | | sequence |
| 7 | GCGGCCCAAAGTAGTACCTGGCCCGAATACTTCCAGTAC | REGN8938 HCDR3 nucleotide |
| | | sequence |
| 8 | AAQSSTWPEYFQY | REGN8938 HCDR3 amino acid |
| | | sequence |
| 9 | GACATCCAGATGACCCAGTCTCCATCCTCCCTGTCTGCATCTGTAGGAGACAGA | REGN8938 LCVR nucleotide |
| | GTCACCATCACTTGCCGGGCAAGTCAGAGCATTAGCAGCTATTTAAATTGGTAT | sequence |
| | CAGCAGAAACCAGGGAAAGCCCCTAAGCTCCTGATCTATGCTGCATCCAGTTTG | |
| | CAAAGTGGGGTCCCGTCAAGGTTCAGTGGCAGTGGATCTGGGACAGATTTCAC | |
| | TCTCACCATCAGCAGTCTGCAACCTGAAGATTTTGCAACTTACTACTGTCAACAG | |
| | AGTTACAGTACCCCTCCGATCACCTTCGGCCAAGGGACACGACTGGAGATTAAA | |
| 10 | DIQMTQSPSSLSASVGDRVTITCRASQSISSYLNWYQQKPGKAPKLLIYAASSLQSG | REGN8938 LCVR amino acid |
| | VPSRFSGSGSGTDFTLTISSLQPEDFATYYCQQSYSTPPITFGQGTRLEIK | sequence |
| 11 | CAGAGCATTAGCAGCTAT | REGN8938 LCDR1 nucleotide |
| | | sequence |
| 12 | QSISSY | REGN8938 LCDR1 amino acid |
| | | sequence |
| 13 | GCTGCATCC | REGN8938 LCDR2 nucleotide |
| | | sequence |
| 14 | AAS | REGN8938 LCDR2 amino acid |
| | | sequence |
| 15 | CAACAGAGTTACAGTACCCCTCCGATCACC | REGN8938 LCDR3 nucleotide |
| | | sequence |
| 16 | QQSYSTPPIT | REGN8938 LCDR3 amino acid |
| | | sequence |
| 17 | CAGGTGCAGCTGGTGGAGTCTGGGGGAGACGTGGTCCAGCCTGGGAGGTCCC | REGN8938 heavy chain |
| | TGAGACTCTCCTGTGCAGCCTCTGGATTCACCTTCAGTTACTATGGCATGCACTG | nucleotide sequence |
| | GGTCCGCCAGCCTCCAGGCAAGGGGCTGGAGTGGGTGACAATTATATCATTTG | |
| | ATGGAAGTCAAAAATACTATGCAGACTCCGTGAAGGGCCGCTTCACCATCTCCA | |
| | GAGACAATTCCAAGAACACGGTTTTTTTGCAAATGAACAGCCTGAGAACAGAG | |
| | GACACGGGTTTCTATTACTGTGCGGCCCAAAGTAGTACCTGGCCCGAATACTTC | |
| | CAGTACTGGGGCCAGGGCACCCTGGTCACCGTCTCCTCAGCCTCCACCAAGGGC | |
| | CCATCGGTCTTCCCCCTGGCGCCCTGCTCCAGGAGCACCTCCGAGAGCACAGCC | |
| | GCCCTGGGCTGCCTGGTCAAGGACTACTTCCCCGAACCGGTGACGGTGTCGTG | |
| | GAACTCAGGCGCCCTGACCAGCGGCGTGCACACCTTCCCGGCTGTCCTACAGTC | |
| | CTCAGGACTCTACTCCCTCAGCAGCGTGGTGACCGTGCCCTCCAGCAGCTTGGG | |
| | CACGAAGACCTACACCTGCAACGTAGATCACAAGCCCAGCAACACCAAGGTGG | |
| | ACAAGAGAGTTGAGTCCAAATATGGTCCCCCATGCCCACCCTGCCCAGCACCTG | |
| | AGTTCCTGGGGGGACCATCAGTCTTCCTGTTCCCCCCAAAACCCAAGGACACTCT | |
| | CATGATCTCCCGGACCCCTGAGGTCACGTGCGTGGTGGTGGACGTGAGCCAGG | |
| | AAGACCCCGAGGTCCAGTTCAACTGGTACGTGGATGGCGTGGAGGTGCATAAT | |
| | GCCAAGACAAAGCCGCGGGAGGAGCAGTTCAACAGCACGTACCGTGTGGTCA | |
| | GCGTCCTCACCGTCCTGCACCAGGACTGGCTGAACGGCAAGGAGTACAAGTGC | |
| | AAGGTCTCCAACAAAGGCCTCCCGTCCTCCATCGAGAAAACCATCTCCAAAGCC | |
| | AAAGGGCAGCCCCGAGAGCCACAGGTGTACACCCTGCCCCCATCCCAGGAGGA | |
| | GATGACCAAGAACCAGGTCAGCCTGACCTGCCTGGTCAAAGGCTTCTACCCCAG | |
| | CGACATCGCCGTGGAGTGGGAGAGCAATGGGCAGCCGGAGAACAACTACAAG | |
| | ACCACGCCTCCCGTGCTGGACTCCGACGGCTCCTTCTTCCTCTACAGCAGGCTCA | |
| | CCGTGGACAAGAGCAGGTGGCAGGAGGGGAATGTCTTCTCATGCTCCGTGATG | |
| | CATGAGGCTCTGCACAACCACTACACACAGAAGTCCCTCTCCCTGTCTCTGGGTA | |
| | AATGA | |
| 18 | QVQLVESGGDVVQPGRSLRLSCAASGFTFSYYGMHWVRQPPGKGLEWVTIISFDG | REGN8938 heavy chain amino |
| | SQKYYADSVKGRFTISRDNSKNTVFLQMNSLRTEDTGFYYCAAQSSTWPEYFQYW | acid sequence |
| | GQGTLVTVSSASTKGPSVFPLAPCSRSTSESTAALGCLVKDYFPEPVTVSWNSGALT | |
| | SGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISK | |
| | AKGQPREPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLSLSLGK | |
| 19 | GACATCCAGATGACCCAGTCTCCATCCTCCCTGTCTGCATCTGTAGGAGACAGA | REGN8938 light chain |
| | GTCACCATCACTTGCCGGGCAAGTCAGAGCATTAGCAGCTATTTAAATTGGTAT | nucleotide sequence |
| | CAGCAGAAACCAGGGAAAGCCCCTAAGCTCCTGATCTATGCTGCATCCAGTTTG | |
| | CAAAGTGGGGTCCCGTCAAGGTTCAGTGGCAGTGGATCTGGGACAGATTTCAC | |
| | TCTCACCATCAGCAGTCTGCAACCTGAAGATTTTGCAACTTACTACTGTCAACAG | |
| | AGTTACAGTACCCCTCCGATCACCTTCGGCCAAGGGACACGACTGGAGATTAAA | |
| | CGAACTGTGGCTGCACCATCTGTCTTCATCTTCCCGCCATCTGATGAGCAGTTGA | |
| | AATCTGGAACTGCCTCTGTTGTGTGCCTGCTGAATAACTTCTATCCCAGAGAGGC | |
| | CAAAGTACAGTGGAAGGTGGATAACGCCCTCCAATCGGGTAACTCCCAGGAGA | |
| | GTGTCACAGAGCAGGACAGCAAGGACAGCACCTACAGCCTCAGCAGCACCCTG | |
| | ACGCTGAGCAAAGCAGACTACGAGAAACACAAAGTCTACGCCTGCGAAGTCAC | |
| | CCATCAGGGCCTGAGCTCGCCCGTCACAAAGAGCTTCAACAGGGGAGAGTGTT | |
| | AG | |
| 20 | DIQMTQSPSSLSASVGDRVTITCRASQSISSYLNWYQQKPGKAPKLLIYAASSLQSG | REGN8938 light chain amino |
| | VPSRFSGSGSGTDFTLTISSLQPEDFATYYCQQSYSTPPITFGQGTRLEIKRTVAAP | acid sequence |
| | SVFIFPPSDEQLKSGTASVVCLLNNFYPREAKVQWKVDNALQSGNSQESVTEQDSKD | |
| | STYSLSSTLTLSKADYEKHKVYACEVTHQGLSSPVTKSFNRGEC | |
| 21 | CAGGTGCAGCTGGTGGAGTCTGGGGGAGGCGTGGTCCAGCCTGGGAGGTCCC | mAb25311 HCVR nucleotide |
| | TGAGACTCTCCTGTGTAGCGTCTGGATTCACCTTCAGTACCTCTGTCATGCACTG | sequence |
| | GGTCCGCCAGGCTCCAGGCAAGGGACTGGAATGGGTGGCAAATATATGGTATG | |
| | ATGGAATCAATAAATTCTATGTAGACTCTGTGAAGGGGCGATTCACCATCTCCA | |
| | GAGACAATTCCAAGAACACCCTGTATCTGCAAATGAACAGCCTGAGAGCCGAG | |
| | GACACGGCTGTATATTACTGTGCGAGAGAGTTTTGGAGTGCCTTTGATCTCTGG | |
| | GGCCAAGGGACAATGGTCACCGTCTCTTCA | |
| 22 | QVQLVESGGGVVQPGRSLRLSCVASGFTFSTSVMHWVRQAPGKGLEWVANIWY | mAb25311 HCVR amino acid |
| | DGINKFYVDSVKGRFTISRDNSKNTLYLQMNSLRAEDTAVYYCAREFWSAFDLWG | sequence |
| | QGTMVTVSS | |
| 23 | GGATTCACCTTCAGTACCTCTGTC | mAb25311 HCDR1 nucleotide |
| | | sequence |
| 24 | GFTFSTSV | mAb25311 HCDR1 amino acid |
| | | sequence |
| 25 | ATATGGTATGATGGAATCAATAAA | mAb25311 HCDR2 nucleotide |
| | | sequence |
| 26 | IWYDGINK | mAb25311 HCDR2 amino acid |
| | | sequence |
| 27 | GCGAGAGAGTTTTGGAGTGCCTTTGATCTC | mAb25311 HCDR3 nucleotide |
| | | sequence |
| 28 | AREFWSAFDL | mAb25311 HCDR3 amino acid |
| | | sequence |
| 29 | GAAATAGTTTTGACACAGAGTCCCGGCACACTGTCACTCTCTCCCGGGGAAAGA | mAb25311 LCVR nucleotide |
| | GCCACCTTGTCATGTAGAGCAAGTCAGTCAGTCTCTAGCTCTTATCTCGCCTGGT | sequence |
| | ACCAGCAGAAGCCGGGACAGGCCCCTAGACTGCTGATCTACGGGGCAAGTTCC | |
| | AGGGCCACCGGAATCCCCGACCGGTTCAGTGGAAGCGGAAGCGGAACCGATTT | |
| | TACTTTGACGATTTCTAGACTGGAGCCAGAGGATTTCGCCGTTTACTATTGTCAA | |
| | CAGTACGGAAGCAGCCCGTGGACGTTTGGCCAGGGCACGAAGGTAGAAATCA | |
| | AG | |
| 30 | EIVLTQSPGTLSLSPGERATLSCRASQSVSSSYLAWYQQKPGQAPRLLIYGASSRAT | mAb25311 LCVR amino acid |
| | GIPDRFSGSGSGTDFTLTISRLEPEDFAVYYCQQYGSSPWTFGQGTKVEIK | sequence |
| 31 | CAGTCAGTCTCTAGCTCTTAT | mAb25311 LCDR1 nucleotide |
| | | sequence |
| 32 | QSVSSSY | mAb25311 LCDR1 amino acid |
| | | sequence |
| 33 | GGGGCAAGT | mAb25311 LCDR2 nucleotide |
| | | sequence |
| 34 | GAS | mAb25311 LCDR2 amino acid |
| | | sequence |
| 35 | CAACAGTACGGAAGCAGCCCGTGGACG | mAb25311 LCDR3 nucleotide |
| | | sequence |
| 36 | QQYGSSPWT | mAb25311 LCDR3 amino acid |
| | | sequence |
| 37 | CAGGTGCAGCTGGTGGAGTCTGGGGGAGGCGTGGTCCAGCCTGGGAGGTCCC | mAb25311 heavy chain |
| | TGAGACTCTCCTGTGTAGCGTCTGGATTCACCTTCAGTACCTCTGTCATGCACTG | nucleotide sequence |
| | GGTCCGCCAGGCTCCAGGCAAGGGACTGGAATGGGTGGCAAATATATGGTATG | |
| | ATGGAATCAATAAATTCTATGTAGACTCTGTGAAGGGGCGATTCACCATCTCCA | |
| | GAGACAATTCCAAGAACACCCTGTATCTGCAAATGAACAGCCTGAGAGCCGAG | |
| | GACACGGCTGTATATTACTGTGCGAGAGAGTTTTGGAGTGCCTTTGATCTCTGG | |
| | GGCCAAGGGACAATGGTCACCGTCTCTTCAGCCTCCACCAAGGGCCCATCGGTC | |
| | TTCCCCCTGGCGCCCTGCTCCAGGAGCACCTCCGAGAGCACAGCCGCCCTGGGC | |
| | TGCCTGGTCAAGGACTACTTCCCCGAACCGGTGACGGTGTCGTGGAACTCAGGC | |
| | GCCCTGACCAGCGGCGTGCACACCTTCCCGGCTGTCCTACAGTCCTCAGGACTC | |
| | TACTCCCTCAGCAGCGTGGTGACCGTGCCCTCCAGCAGCTTGGGCACGAAGACC | |
| | TACACCTGCAACGTAGATCACAAGCCCAGCAACACCAAGGTGGACAAGAGAGT | |
| | TGAGTCCAAATATGGTCCCCCATGCCCACCCTGCCCAGCACCTGAGTTCCTGGG | |
| | GGGACCATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCCCG | |
| | AGGTCCAGTTCAACTGGTACGTGGATGGCGTGGAGGTGCATAATGCCAAGACA | |
| | AAGCCGCGGGAGGAGCAGTTCAACAGCACGTACCGTGTGGTCAGCGTCCTCAC | |
| | CGTCCTGCACCAGGACTGGCTGAACGGCAAGGAGTACAAGTGCAAGGTCTCCA | |
| | ACAAAGGCCTCCCGTCCTCCATCGAGAAAACCATCTCCAAAGCCAAAGGGCAGC | |
| | CCCGAGAGCCACAGGTGTACACCCTGCCCCCATCCCAGGAGGAGATGACCAAG | |
| | AACCAGGTCAGCCTGACCTGCCTGGTCAAAGGCTTCTACCCCAGCGACATCGCC | |
| | GTGGAGTGGGAGAGCAATGGGCAGCCGGAGAACAACTACAAGACCACGCCTC | |
| | CCGTGCTGGACTCCGACGGCTCCTTCTTCCTCTACAGCAGGCTCACCGTGGACA | |
| | AGAGCAGGTGGCAGGAGGGGAATGTCTTCTCATGCTCCGTGATGCATGAGGCT | |
| | CTGCACAACCACTACACACAGAAGTCCCTCTCCCTGTCTCTGGGTAAATGA | |
| 38 | QVQLVESGGGVVQPGRSLRLSCVASGFTFSTSVMHWVRQAPGKGLEWVANIWY | mAb25311 heavy chain |
| | DGINKFYVDSVKGRFTISRDNSKNTLYLQMNSLRAEDTAVYYCAREFWSAFDLWG | amino acid sequence |
| | QGTMVTVSSASTKGPSVFPLAPCSRSTSESTAALGCLVKDYFPEPVTVSWNSGALTS | |
| | GVHTFPAVLQSSGLYSLSSVVTVPSSSLGTKTYTCNVDHKPSNTKVDKRVESKYGPP | |
| | CPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISK | |
| | AKGQPREPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLSLSLGK | |
| 39 | GAAATAGTTTTGACACAGAGTCCCGGCACACTGTCACTCTCTCCCGGGGAAAGA | mAb25311 light chain |
| | GCCACCTTGTCATGTAGAGCAAGTCAGTCAGTCTCTAGCTCTTATCTCGCCTGGT | nucleotide sequence |
| | ACCAGCAGAAGCCGGGACAGGCCCCTAGACTGCTGATCTACGGGGCAAGTTCC | |
| | AGGGCCACCGGAATCCCCGACCGGTTCAGTGGAAGCGGAAGCGGAACCGATTT | |
| | TACTTTGACGATTTCTAGACTGGAGCCAGAGGATTTCGCCGTTTACTATTGTCAA | |
| | CAGTACGGAAGCAGCCCGTGGACGTTTGGCCAGGGCACGAAGGTAGAAATCA | |
| | AGCGAACTGTGGCTGCACCATCTGTCTTCATCTTCCCGCCATCTGATGAGCAGTT | |
| | GAAATCTGGAACTGCCTCTGTTGTGTGCCTGCTGAATAACTTCTATCCCAGAGA | |
| | GGCCAAAGTACAGTGGAAGGTGGATAACGCCCTCCAATCGGGTAACTCCCAGG | |
| | AGAGTGTCACAGAGCAGGACAGCAAGGACAGCACCTACAGCCTCAGCAGCACC | |
| | CTGACGCTGAGCAAAGCAGACTACGAGAAACACAAAGTCTACGCCTGCGAAGT | |
| | CACCCATCAGGGCCTGAGCTCGCCCGTCACAAAGAGCTTCAACAGGGGAGAGT | |
| | GTTAG | |
| 40 | EIVLTQSPGTLSLSPGERATLSCRASQSVSSSYLAWYQQKPGQAPRLLIYGASSRAT | mAb25311 light chain |
| | GIPDRFSGSGSGTDFTLTISRLEPEDFAVYYCQQYGSSPWTFGQGTKVEIKRTVAAP | amino acid sequence |
| | SVFIFPPSDEQLKSGTASVVCLLNNFYPREAKVQWKVDNALQSGNSQESVTEQDSKD | |
| | STYSLSSTLTLSKADYEKHKVYACEVTHQGLSSPVTKSFNRGEC | |
| 41 | MAAPVPWACCAVLAAAAAVVYAQRHSPQEAPHVQYERLGSDVTLPCGTANWDA | Human CNTFR receptor |
| | AVTWRVNGTDLAPDLLNGSQLVLHGLELGHSGLYACFHRDSWHLRHQVLLHVGLP | subunit alpha (UniProt |
| | PREPVLSCRSNTYPKGFYCSWHLPTPTYIPNTFNVTVLHGSKIMVCEKDPALKNRCH | Accession No. P26992) |
| | IRYMHLFSTIKYKVSISVSNALGHNATAITFDEFTIVKPDPPENVVARPVPSNPRRL | |
| | EVTWQTPSTWPDPESFPLKFFLRYRPLILDQWQHVELSDGTAHTITDAYAGKEYIIQ | |
| | VAAKDNEIGTWSDWSVAAHATPWTEEPRHLTTEAQAAETTTSTTSSLAPPPTTKICD | |
| | PGELGSGGGPSAPFLVSVPITLALAAAAATASSLLI | |
| 42 | QRHSPQEAPHVQYERLGSDVTLPCGTANWDAAVTWRVNGTDLAPDLLNGSQLVL | Human CNTFR-mmh (mmh |
| | HGLELGHSGLYACFHRDSWHLRHQVLLHVGLPPREPVLSCRSNTYPKGFYCSWHLP | tag is underlined) |
| | TPTYIPNTFNVTVLHGSKIMVCEKDPALKNRCHIRYMHLFSTIKYKVSISVSNALGH | |
| | ANTAITFDEFTIVKPDPPENVVARPVPSNPRRLEVTWQTPSTWPDPESFPLKFFLRY | |
| | RPLILDQWQHVELSDGTAHTITDAYAGKEYIIQVAAKDNEIGTWSDWSVAAHATPWT | |
| | EEPRHLTTEAQAAETTTSTTSSLAPPPTTKICDPGELGSEQKLISEEDLGGEQKLIS | |
| | EEDLHHHHHH | |
| 43 | QRHSPQEAPHVQYERLGSDVTLPCGTANWDAAVTWRVNGTDLAPDLLNGSQLVL | Monkey CNTFR-mmh (mmh |
| | RGLELGHSGLYACFHRDSWHLRHQVLLHVGLPPREPVLSCRSNTYPKGFYCSWHLP | tag is underlined) |
| | TPTYIPNTFNVTVLHGSKIMVCEKDPALKNRCHIRYMHLFSTIKYKVSISVSNALGH | |
| | NATAITFDEFTIVKPDPPENVVARPVPSNPRRLEVTWQIPSTWPDPESFPLKFFLRY | |
| | RPLILDQWQHVELSDGTAHTITDAYAGKEYIIQVAAKDNEIGTWSDWSVAAHATPWT | |
| | EEPRHLTTEAQAAETTTSTTSSLAPPPTTKICDPGELGSEQKLISEEDLGGEQKLI | |
| | SEEDLHHHHHH | |
| 44 | QKHSPQEAPHVQYERLGADVTLPCGTASWDAAVTWRVNGTDLAPDLLNGSQLIL | Mouse CNTFR-mmh (mmh tag |
| | RSLELGHSGLYACFHRDSWHLRHQVLLHVGLPPREPVLSCRSNTYPKGFYCSWHLP | is underlined) |
| | TPTYIPNTFNVTVLHGSKIMVCEKDPALKNRCHIRYMHLFSTIKYKVSISVSNALGH | |
| | NTTAITFDEFTIVKPDPPENVVARPVPSNPRRLEVTWQTPSTWPDPESFPLKFFLRY | |
| | RPLILDQWQHVELSDGTAHTITDAYAGKEYIIQVAAKDNEIGTWSDWSVAAHATPWT | |
| | EEPRHLTTEAQAPETTTSTTSSLAPPPTTKICDPGELGSEQKLISEEDLGGEQKLIS | |
| | EEDLHHHHHH | |
| 45 | QRHSPQEAPHVQYERLGSDVTLPCGTANWDAAVTWRVNGTDLAPDLLNGSQLVL | Human CNTFR-mFc (mFc tag |
| | HGLELGHSGLYACFHRDSWHLRHQVLLHVGLPPREPVLSCRSNTYPKGFYCSWHLP | is underlined) |
| | TPTYIPNTFNVTVLHGSKIMVCEKDPALKNRCHIRYMHLFSTIKYKVSISVSNALGH | |
| | NATAITFDEFTIVKPDPPENVVARPVPSNPRRLEVTWQTPSTWPDPESFPLKFFLRY | |
| | RPLILDQWQHVELSDGTAHTITDAYAGKEYIIQVAAKDNEIGTWSDWSVAAHATPWT | |
| | EEPRHLTTEAQAAETTTSTTSSLAPPPTTKICDPGELGSEPRGPTIKPCPPCKCPAP | |
| | NLLGGPSVFIFPPKIKDVLMISLSPIVTCVVVDVSEDDPDVQISWFVNNVEVHTAQT | |
| | QTHREDYNSTLRVVSALPIQHQDWMSGKEFKCKVNNKDLPAPIERTISKPKGSVRAP | |
| | QVYVLPPPEEEMTKKQVTLTCMVTDFMPEDIYVEWTNNGKTELNYKNTEPVLDSDG | |
| | SYFMYSKLRVEKKNWVERNSYSCSVVHEGLHNHHTTKSFSRTPGK | |
| 46 | QRHSPQEAPHVQYERLGSDVTLPCGTANWDAAVTWRVNGTDLAPDLLNGSQLVL | Human CNTFR-hFc (hFc tag |
| | HGLELGHSGLYACFHRDSWHLRHQVLLHVGLPPREPVLSCRSNTYPKGFYCSWHLP | is underlined) |
| | TPTYIPNTFNVTVLHGSKIMVCEKDPALKNRCHIRYMHLFSTIKYKVSISVSNALGH | |
| | NATAITFDEFTIVKPDPPENVVARPVPSNPRRLEVTWQTPSTWPDPESFPLKFFLRY | |
| | RPLILDQWQHVELSDGTAHTITDAYAGKEYIIQVAAKDNEIGTWSDWSVAAHATPWT | |
| | EEPRHLTTEAQAAETTTSTTSSLAPPPTTKICDPGELGSDKTHTCPPCPAPELLGGP | |
| | SVFLFPPKPKDTLMISRTPEVTCVVVDVSHEDPEVKFNWYVDGVEVHNAKTKPREEQ | |
| | YNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| 47 | QRHSPQEAPHVQYERLGSDVTLPCGTANWDAAVTWRVNGTDLAPDLLNGSQLVL | hCNTFR(Q23-V105)/mCNTFR |
| | HGLELGHSGLYACFHRDSWHLRHQVLLHVGLPPREPVLSCRSNTYPKGFYCSWHLP | (G106-S342).mmh (mmh tag |
| | TPTYIPNTENVTVLHGSKIMVCEKDPALKNRCHIRYMHLFSTIKYKVSISVSNALGH | is underlined) |
| | NTTAITFDEFTIVKPDPPENVVARPVPSNPRRLEVTWQTPSTWPDPESFPLKFFLRY | |
| | RPLILDQWQHVELSDGTAHTITDAYAGKEYHIQVAAKDNEIGTWSDWSVAAHATPWT | |
| | EEPRHLTTEAQAPETTTSTTSSLAPPPTTKICDPGELGSEQKLISEEDLGGEQKLIS | |
| | EEDLHHHHHH | |
| 48 | QRHSPQEAPHVQYERLGSDVTLPCGTANWDAAVTWRVNGTDLAPDLLNGSQLVL | hCNTFR (Q23-D205)/ |
| | HGLELGHSGLYACFHRDSWHLRHQVLLHVGLPPREPVLSCRSNTYPKGFYCSWHLP | mCNTFR(P206-S342).mmh |
| | TPTYIPNTFNVTVLHGSKIMVCEKDPALKNRCHIRYMHLFSTIKYKVSISVSNALGH | (mmh tag is underlined) |
| | NATAITFDEFTIVKPDPPENVVARPVPSNPRRLEVTWQTPSTWPDPESFPLKFFLRY | |
| | RPLILDQWQHVELSDGTAHTITDAYAGKEYIIQVAAKDNEIGTWSDWSVAAHATPWT | |
| | EEPRHLTTEAQAPETTTSTTSSLAPPPTTKICDPGELGSEQKLISEEDLGGEQKLIS | |
| | EEDLHHHHHH | |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of all patents and non-patent literature cited herein are expressly incorporated in their entirety by reference.

What is claimed is:

1. An isolated monoclonal antibody or an antigen-binding fragment thereof that specifically binds to human ciliary neurotrophic factor receptor (CNTFR), wherein the antibody or antigen-binding fragment thereof comprises:
(a) three heavy chain CDRs (HCDR1, HCDR2, and HCDR3) contained within a heavy chain variable region (HCVR) amino acid sequence of SEQ ID NO: 2 or an HCVR having at least 85%, at least 90%, at least 95%, at least 97%, at least 98%, or at least 99% sequence identity to SEQ ID NO: 2; and three light chain CDRs (LCDR1, LCDR2, and LCDR3) contained within a light chain variable region (LCVR) amino acid sequence of SEQ ID NO: 10 or an LCVR having at least 85%, at least 90%, at least 95%, at least 97%, at least 98%, or at least 99% sequence identity to SEQ ID NO: 10; or
(b) three heavy chain CDRs (HCDR1, HCDR2, and HCDR3) contained within a heavy chain variable region (HCVR) amino acid sequence of SEQ ID NO: 22 or an HCVR having at least 85%, at least 90%, at least 95%, at least 97%, at least 98%, or at least 99% sequence identity to SEQ ID NO: 22; and three light chain CDRs (LCDR1, LCDR2, and LCDR3) contained within a light chain variable region (LCVR) amino acid sequence of SEQ ID NO: 30 or an LCVR having at least 85%, at least 90%, at least 95%, at least 97%, at least 98%, or at least 99% sequence identity to SEQ ID NO: 30.

2. The isolated monoclonal antibody or antigen-binding fragment thereof of claim 1, wherein the antibody or antigen-binding fragment thereof binds to dimeric human CNTFR with a K_{D} of less than 10 nM as measured by surface plasmon resonance at 25° C.

3. The isolated monoclonal antibody or antigen-binding fragment thereof of claim 1, wherein the antibody:
(i) binds an epitope in the region of Q23-V105 of human CNTFR;
(ii) binds an epitope in the region of V105-D205 of human CNTFR;
(iii) cross-reacts with cynomolgus monkey CNTFR; and/or
(iv) is a fully human monoclonal antibody.

4. The isolated monoclonal antibody or antigen-binding fragment thereof of claim 1, wherein the antibody or antigen-binding fragment thereof comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO: 12, an LCDR2 comprising the amino acid sequence of SEQ ID NO:14, and an LCDR3 comprising the amino acid sequence of SEQ ID NO:16.

5. The isolated monoclonal antibody or antigen-binding fragment thereof of claim 1, wherein the antibody or antigen-binding fragment thereof comprises a heavy chain variable region (HCVR) amino acid sequence of SEQ ID NO: 2.

6. The isolated monoclonal antibody or antigen-binding fragment thereof of claim 1, wherein the antibody or antigen-binding fragment thereof comprises a light chain variable region (LCVR) amino acid sequence of SEQ ID NO: 10.

7. The isolated monoclonal antibody or antigen-binding fragment thereof of claim 1, wherein the antibody or antigen-binding fragment thereof comprises:
an HCVR comprising the amino acid sequence of SEQ ID NO: 2 and an LCVR comprising the amino acid sequence of SEQ ID NO: 10.

8. The isolated monoclonal antibody or antigen-binding fragment thereof of claim 1, wherein the antibody or antigen-binding fragment thereof comprises:
(a) a heavy chain comprising an amino acid sequence having at least 85% sequence identity to the amino acid sequence of SEQ ID NO: 18 and a light chain comprising an amino acid sequence having at least 85% sequence identity to the amino acid sequence of SEQ ID NO: 20.

9. The isolated monoclonal antibody or antigen-binding fragment thereof of claim 8, wherein the antibody or antigen-binding fragment thereof comprises a heavy chain comprising the amino acid sequence of SEQ ID NO: 18 and a light chain comprising the amino acid sequence of SEQ ID NO: 20.

10. The isolated monoclonal antibody or antigen-binding fragment thereof of claim 1, wherein the antibody or antigen-binding fragment thereof comprises
an HCDR1 comprising the amino acid sequence of SEQ ID NO:24, an HCDR2 comprising the amino acid sequence of SEQ ID NO:26, an HCDR3 comprising the amino acid sequence of SEQ ID NO:28, an LCDR1 comprising the amino acid sequence of SEQ ID NO:32, an LCDR2 comprising the amino acid sequence of SEQ ID NO:34, and an LCDR3 comprising the amino acid sequence of SEQ ID NO:36.

11. The isolated monoclonal antibody or antigen-binding fragment thereof of claim 1, wherein the antibody or antigen-binding fragment thereof comprises a heavy chain variable region (HCVR) amino acid sequence of SEQ ID NO: 22.

12. The isolated monoclonal antibody or antigen-binding fragment thereof of claim 1, wherein the antibody or antigen-binding fragment thereof comprises a light chain variable region (LCVR) amino acid sequence of SEQ ID NO: 30.

13. The isolated monoclonal antibody or antigen-binding fragment thereof of claim 1, wherein the antibody or antigen-binding fragment thereof comprises:
an HCVR comprising the amino acid sequence of SEQ ID NO: 22 and an LCVR comprising the amino acid sequence of SEQ ID NO: 30.

14. The isolated monoclonal antibody or antigen-binding fragment thereof of claim 1, wherein the antibody or antigen-binding fragment thereof comprises:
a heavy chain comprising an amino acid sequence having at least 85% sequence identity to the amino acid sequence of SEQ ID NO: 38 and a light chain comprising an amino acid sequence having at least 85% sequence identity to the amino acid sequence of SEQ ID NO: 40.

15. The isolated monoclonal antibody or antigen-binding fragment thereof of claim 14, wherein the antibody or antigen-binding fragment thereof comprises:
a heavy chain comprising the amino acid sequence of SEQ ID NO: 38 and a light chain comprising the amino acid sequence of SEQ ID NO: 40.

16. A pharmaceutical composition comprising the monoclonal antibody or antigen-binding fragment thereof of claim 1 and a pharmaceutically acceptable carrier.

17. A nucleic acid molecule comprising a nucleotide sequence encoding the monoclonal antibody or antigen-binding fragment thereof of claim 1.

18. A nucleic acid molecule comprising one or more nucleotide sequences set forth in Table 14.

19. An expression vector comprising a nucleic acid molecule encoding an HCVR or an LCVR of claim 1.

20. A host cell comprising the expression vector of claim 19.

21. A method of producing an anti-CNTFR antibody or antigen-binding fragment thereof, the method comprising introducing the expression vector of claim 19 into a host cell, growing the host cell under conditions permitting production of the antibody or antigen-binding fragment thereof, and recovering the antibody or antigen-binding fragment that is produced.

22. An expression vector comprising a nucleic acid molecule encoding an HCVR or an LCVR of claim 1.

23. A host cell comprising the expression vector of claim 22.

24. A method of producing an anti-CNTFR antibody or antigen-binding fragment thereof, the method comprising introducing the expression vector of claim 22 into a host cell, growing the host cell under conditions permitting production of the antibody or antigen-binding fragment thereof, and recovering the antibody or antigen-binding fragment that is produced.

25. A method of treating a disease, disorder, or condition associated with CNTFR expression or activity, the method comprising administering the monoclonal antibody or antigen-binding fragment thereof of claim 1 to a subject in need thereof.

26. The method of claim 25, wherein the disease, disorder, or condition is a metabolic disease.

27. The method of claim 25, wherein the disease, disorder, or condition is a neurological or neurodegenerative disease.

28. A method of inhibiting CNTF-mediated activation of cells expressing CNFTR, the method comprising contacting the cells with the monoclonal antibody or antigen-binding fragment thereof of claim 1.
